(19) United States
(12) Patent Application Publication
(10) Pub. No.: US 2013/0243801 A1
(43) Pub. Date: Sep. 19, 2013

(54) EPITOPE THERAPY FOR INFECTIOUS DISEASES

(71) Applicants: Joel D. Ernst, New York, NY (US); Tyler D. Bold, Boston, MA (US)

(72) Inventors: Joel D. Ernst, New York, NY (US); Tyler D. Bold, Boston, MA (US)

(73) Assignee: NEW YORK UNIVERSITY, New York, NY (US)

(21) Appl. No.: 13/811,915

(22) PCT Filed: Aug. 18, 2011
(86) PCT No.: PCT/US2011/048279

§ 371 Date: May 29, 2013

Related U.S. Application Data
(60) Provisional application No. 61/375,892, filed on Aug. 23, 2010.

(51) Int. Cl. A61K 39/04

(52) U.S. Cl. CPC A61K 39/04
USPC 424/185.1; 424/184.1; 424/197.11; 424/248.1; 530/326

(57) ABSTRACT

The present invention relates to a method of treating a subject for an infectious disease. This method involves selecting a subject infected with an infectious disease-causing agent. The infectious disease-causing agent elicits, in the selected subject, an immune response that is insufficient to cure the selected subject of the infectious disease. The method also involves administering to the selected subject a synthetic peptide representing an antigen epitope associated with an immune response to the infectious agent. The immune response to the synthetic peptide is sufficient to treat the selected subject for the infectious disease.

This application claims the benefit of U.S. Provisional Patent Application Ser. No. 61/375,892, filed Aug. 23, 2010, which is hereby incorporated by reference in its entirety.

This invention was made with government support under National Institutes of Health Grant Nos. R01 AI051242 (NIAID) and F30 HL096342 (NHLBI). The government has certain rights in the invention.

### FIELD OF THE INVENTION

This invention relates to epitope therapy for infectious diseases.

### BACKGROUND OF THE INVENTION

Even though its etiologic agent was discovered over 125 years ago, tuberculosis remains a global scourge, killing 1.7 million people in 2009, at least ¾ of whom were immunocompetent (“Global Tuberculosis Control: Epidemiology, Strategy, Fnancing,” WHO Report, 2009). Long-term persistence of Mycobacterium tuberculosis, which resides principally in phagocytic cells within the lungs, results in a chronic infection despite the presence of an apparently appropriate adaptive immune response. In mice infected with virulent M. tuberculosis, the early phase of infection proceeds with unchecked bacterial growth until day 17-21 post-infection, when adaptive immunity finally exerts control of bacterial growth in the lungs. Control of infection in both humans and mice critically depends on M. tuberculosis-specific CD4⁺ Th1 cell responses, which include production of IFN-γ (Cooper, “T Cells in Mycobacterial Infection and Disease,” Curr. Opin. Immunol. 21:378-84 (2000); Wolf et al., “Initiation of the Adaptive Immune Response to Mycobacterium tuberculosis Depends on Antigen Production in the Local Lymph Node, not the Lungs,” J. Exp. Med. 205:105-15 (2008)). However, adaptive immune responses do not eradicate the infection.

Several potential mechanisms may account for the failure of adaptive immune responses to eradicate the bacteria in tuberculosis. Generation of M. tuberculosis-specific CD4⁺ effector T cells is delayed compared with responses to other pathogens (Reiley et al., “ESAT-6-Specific CD4 T Cell Responses to Aerosol Mycobacterium tuberculosis Infection are Initiated in the Mediastinal Lymph Nodes,”Proc. Natl. Acad. Sci. 105:10961-6 (2008); Wolf et al., “Initiation of the Adaptive Immune Response to Mycobacterium tuberculosis Depends on Antigen Production in the Local Lymph Node, not the Lungs,” J. Exp. Med. 205:105-15 (2008)). In addition, certain individuals, or strains of mice, may develop inappropriate (e.g., Th2) (Flynn, et al., “IL-12 Increases Resistance of BALB/c Mice to Mycobacterium tuberculosis Infection,” J. Immunol. 155:2515-24 (1995); Wangoo et al., “Contribution of Th1 and Th2 Cells to Protection and Pathology in Experimental Models of Granulomatous Lung Disease,” J. Immunol. 166:3432-9 (2001)) or imbalanced effector phenotypes such as Th1/Th17 (Chen et al., “Reduced Th17 Response in Patients with Tuberculosis Correlates with IL-6R Expression on CD4⁺ T Cells,” Am. J. Respir. Crit. Care Med. 181:734-42 (2010)) in response to infection. However, even in humans or mice that develop Th1 responses, a failure of CD4⁺ effector T cells to recognize infected cells may preclude their optimal activation and limit induction of effector functions in the lungs. Prevention of effector T cell activation could result from impaired antigen presentation by lung APCs containing M. tuberculosis (Baena & Porcelli, “Evasion and Subversion of Antigen Presentation by Mycobacterium tuberculosis,” Tissue Antigens 74:189-204 (2009); Divangahi et al., “Eicosanoid Pathways Regulate Adaptive Immunity to Mycobacterium tuberculosis,” Nat. Immunol. 11:751-758 (2010); Harding & Boom, “Regulation of Antigen Presentation by Mycobacterium tuberculosis: A Role for Toll-like Receptors,” Nat. Rev. Microbiol. 8:296-307 (2010)) or because the antigens that effector T cells recognize are not expressed or otherwise available in the lungs. Furthermore, host regulatory mechanisms that limit immune pathology, such as T regulatory cells (Scott-Browne et al., “Expansion and Function of Foxp3-Expressing T Regulatory Cells During Tuberculosis,” J. Exp. Med. 204:2159-69 (2007)), production of inhibitory cytokines (Turner et al., “In vivo IL-10 Production Reactivates Chronic Pulmonary Tuberculosis in C57BL/6 Mice,” J. Immunol. 169:6343-51 (2002)) and, possibly, onset of T cell exhaustion (Yi et al., “T-cell Exhaustion: Characteristics, Causes and Conversion,” Immunology 129:474-81 (2010); Reiley et al., “Distinct Functions of Antigen-Specific CD4 T Cells During Murine Mycobacterium tuberculosis Infection,” Proc. Natl. Acad. Sci. 107:19408-13 (2010)) may inhibit the activity of effector T cells at the site of infection. Finally, even when CD4⁺ effector T cells are activated, the efficacy of these responses may be limited by the impaired ability of infected cells to respond to IFN-γ (Banaiee et al., “Potent Inhibition of Macrophage Responses to IFN-Gamma by Live Virulent Mycobacterium tuberculosis is Independent of Mature Mycobacterial Lipoproteins but Dependent on TLR2,” J. Immunol. 176:3019-27 (2006); Pai et al., “Inhibition of IFN-Gamma-Induced Class II Transactivator Expression by a 19-kDa Lipoprotein from Mycobacterium tuberculosis: A Potential Mechanism for Immune Evasion,” J. Immunol. 171:175-84 (2003); Ting et al., “Mycobacterium tuberculosis Inhibits IFN-Gamma Transcriptional Responses Without Inhibiting Activation of STAT1,” J. Immunol. 163:3898-906 (1999)), induce phagosome maturation (Clemens & Horwitz, “Characterization of the Mycobacterium tuberculosis Phagosome and Evidence that Phagosomal Maturation is Inhibited,” J. Exp. Med. 181:257-70 (1995); Rohde et al., “Mycobacterium tuberculosis and the Environment Within the Phagosome,” Immunol. Rev. 219:37-54 (2007)), or undergo apoptosis (Divangahi et al., “Eicosanoid Pathways Regulate Adaptive Immunity to Mycobacterium tuberculosis,” Nat. Immunol. 11:751-758 (2010); Hinchey et al., “Enhanced Priming of Adaptive Immunity by a Proapoptotic Mutant of Mycobacterium tuberculosis,” J. Clin. Invest. 117:2279-88 (2007); Miller et al., “The Type I NADH Dehydrogenase of Mycobacterium tuberculosis Counters Phagosomal NOX2 Activity to Inhibit TNF-Alpha-Mediated Host Cell Apoptosis,” PLoS Pathog. 6:e1000864 (2010); Velmurugan et al., “Mycobacterium tuberculosis nuoG is a Virulence Gene that Inhibits Apoptosis of Infected Host Cells,” PLoS Pathog. 3:e110 (2007)). Understanding the contribution of each of these potential mechanisms limiting adaptive immunity to M. tuberculosis is essential to vaccine design and other immunologic approaches to tuberculosis prevention and therapy.

The present invention is directed to overcoming these and other limitations in the art.

### SUMMARY OF THE INVENTION

One aspect of the present invention relates to a method of treating a subject for an infectious disease. This method involves selecting a subject infected with an infectious disease-causing agent. The infectious disease-causing agent elicits, in the selected subject, an immune response that is insufficient to cure the selected subject of the infectious disease. The method also involves administering to the selected subject a synthetic peptide representing an antigen epitope associated with an immune response to the infectious agent. The immune response to the synthetic peptide is sufficient to treat the selected subject for the infectious disease.

It is here reported that CD4⁺ effector T cells are activated at submaximal and suboptimal frequencies in the lungs during M. tuberculosis infection, that this is due in part to bacterial modulation of antigen expression, and that increasing the availability of a single epitope results in improved immune control of M. tuberculosis.

Mycobacterium tuberculosis causes persistent infection even in human or animal hosts that develop antigen specific CD4⁺ and CD8⁺ T cell responses. To understand this phenomenon, the hypothesis that the CD4⁺ effector T cells that are generated in response to M. tuberculosis infection fail to encounter their antigens at the site of infection in the lungs was tested. Using mice infected with M. tuberculosis, and an assay of in vivo antigen-dependent activation of CD4⁺ T cells, it was found that both polyclonal CD4⁺ and T cell receptor transgenic CD4⁺ T cells specific for antigen 85B peptide 25 are activated at low frequencies in the lungs. It was found that this is due in part to down regulation of antigen gene expression by M. tuberculosis, as forced expression of the antigen gene resulted in higher frequency activation of CD4⁺ T cells, as well as CD4⁺ T cell-dependent reduction in bacterial burdens and prolonged survival of infected mice. It was also found that administration of antigen 85B peptide 25, which is recognized by a high proportion of M. tuberculosis-specific CD4⁺ T cells, reduced the bacterial burden in the lungs, indicating that stimulation of existing antigen specific CD4⁺ T cells may be a promising approach to therapy of tuberculosis (“TB”).

### BRIEF DESCRIPTION OF THE DRAWINGS

FIGS. 1A-B show low frequency of IFN-γ-producing endogenous CD4⁺ T cells in the lungs of M. tuberculosis-infected mice. FIG. 1A is a series of flow cytometry dot plots showing frequency of IFN-γ expression by endogenous, polyclonal CD4⁺ T cells from the lungs of M. tuberculosis-infected mice throughout infection, assayed by intracellular cytokine staining without ex vivo restimulation. Flow cytometry dot plots show lung CD4⁺ cells from a representative mouse at the indicated time point post-infection. Values indicate the proportion of cells expressing IFN-γ in the CD4⁺ population for each mouse. FIG. 1B is a graph showing mean frequency of IFN-γ′ cells among lung CD4⁺ T cells for each group of 4 mice at each time point post-infection, assayed by intracellular cytokine staining without ex vivo restimulation. Asterisks indicate statistical significance of differences in frequency of T cell activation observed between adjacent time points * p<0.05; ** p<0.005.

FIGS. 2A-D demonstrate that P25TCRTh1 cells produce IFN-γ in response to M. tuberculosis Ag85B peptide 25. FIG. 2A is a pair of flow cytometry dot plots demonstrating that P25TCRTh1 cells were restimulated in vitro with C57BL/6 splenocytes in the presence or absence of peptide 25 and analyzed by flow cytometry for intracellular IFN-γ. FIG. 2B is a photograph showing that CFP-P25TCRTh1 cells traffic to the lung parenchyma of M. tuberculosis infected mice. Th1 effector cells were transferred on day 25, and lungs were harvested on day 28 post infection. CFP-P25TCRTh1 cells (light blue-green) are found in interstitial regions with a high density of DAPI-stained nuclei, typical of the aggregates of macrophages, dendritic cells, and lymphocytes observed at this stage of infection. Scale bar: 50 μm. FIG. 2C is a series of flow cytometry dot plots showing that on day 18 post-infection, mice infected with either H37Rv (w.t.) or ΔAg85B M. tuberculosis that received P25TCRTh1 cells by adoptive transfer. Lung cells were harvested 72 hours later (day 21). Transferred P25TCRTh1 (CD45.2⁺) cells were analyzed by flow cytometry for intracellular IFN-γ. One group of mice received intravenous treatment with Ag85B peptide 25 6 hours prior to lung cell harvest. Flow cytometry dot plots from in vivo experiments show a representative of four mice per group. FIG. 2D is a pair of graphs showing day 21 post-infection with either H37Rv or ΔAg85B: mean percentage from four individual mice of P25TCRTh1 or endogenous CD4⁺ T cells expressing IFN-γ with or without in vivo administration of Ag85B peptide 25.

FIGS. 3A-C show peptide 25-specific T cell activation and fbpB expression decrease during chronic infection. FIG. 3A is a series of dot plots showing frequency of IFN-γ production by adoptively transferred P25TCRTh1 (CD45.2⁺) CD4⁺ cells in the lungs of M. tuberculosis-infected mice throughout infection, assayed by intracellular cytokine staining without ex vivo restimulation. The flow cytometry dot plots show lung P25TCRTh1 cells which were adoptively transferred 3 days prior to the indicated time point post-infection. Values indicate the proportion of cells expressing IFN-γ among the CD45.2⁺, CD4⁺ population for each mouse. FIG. 3B is a series of graphs showing CFSE proliferation profile of naïve P25TCR-tg CD4⁺ T cells transferred into M. tuberculosis-infected wild-type recipients on days 11, 17, or 35 post-infection. Mediastinal lymph node cells were isolated 7 days after adoptive transfer (days 18, 24, or 48 post-infection) and analyzed by flow cytometry for CFSE dilution to measure proliferation. Histograms are representative of four individual mice per time point. FIG. 3C is a graph showing the mean percentage of P25TCRTh1 cells from four individual mice expressing IFN-γ at each time point post-infection compared with the expression of M. tuberculosis fbpB mRNA as infection progresses to chronic phase. Copy number of fbpB mRNA for four individual mice at each time point was determined by RT-qPCR and is normalized to constitutively expressed 16S rRNA.

FIGS. 4A-F demonstrate that forced expression of fbpB enhances T cell activation and impairs bacterial persistence during chronic infection. FIG. 4A is a graph showing expression of fbpB mRNA, normalized to 16S rRNA by H37Rv and CPE85B throughout in vivo infection, determined by RT-qPCR of bacteria in lungs. Data points indicate the mean (±SEM) of 4 mice per time point. FIG. 4B is a bar graph relating to bacterial population size of H37Rv or CPE85B in vitro culture before and after stationary liquid incubation to induce expression of hspXp:fbpB. Columns represent the mean (±SEM) population size of three cultures for each strain. Western blot shows Ag85B protein secreted into culture supernatants during stationary culture. FIG. 4C is a bar graph showing activation of P25TCRTh1 cells in vitro by bone marrow derived DCs infected with H37Rv or CPE85B, measured by IFN-γ ELISA. Columns represent the mean (±SEM) of 3 wells at the indicated infected DC:T cell ratio. FIGS. 4D and 4E relate to activation of P25TCRTh1 cells during mouse infection with H37Rv or CPE85B. P25TCRTh1 cells were transferred into infected mice on either day 18 or 39 post-infection. 3 days after adoptive transfer (day 21 or 42), lung cells were analyzed by flow cytometry for intracellular IFN-γ without ex vivo restimulation. In FIG. 4D, data points indicate the frequency of cells expressing IFN-γ among CD45.2⁺, CD4⁺ lung cells at each time point. In FIG. 4E, flow cytometry plots show lung P25TCRTh1 cells from a representative mouse at the indicated time point post-infection. Values indicate the proportion of IFN-γ′ cells among CD45.2⁺, CD4⁺ population. FIG. 4F is a graph showing bacterial population size throughout mouse infection with H37Rv or CPE85B. Data points indicate the mean (±SEM) of 4 mice per time point.

FIGS. 5A-B show that forced expression of fbpB impairs M. tuberculosis in a CD4⁺ T cell dependent manner. FIG. 5A shows survival of C57BL/6 and CD4⁺ T cell-deficient MHCII KO mice after aerosol infection with H37Rv or CPE85B. N≧5 mice for each group. FIG. 5B shows bacterial population size in lungs of mice infected with H37Rv or CPE85B after CD4⁺ T cell depletion with monoclonal anti-CD4 antibody GK1.5. Antibody treatment was started on day 28 post-infection and continued every 6 days until day 50. Data points indicate the mean (±SEM) bacterial burden in 4 mice in each infection group at each time point. Asterisks indicate statistical significance between groups of mice at neighboring time points within one infection group; * p<0.05; ** p<0.005.

FIGS. 6A-B show that treatment with peptide 25 transiently enhances CD4⁺ T cell responses and reduces bacterial burden. FIG. 6A shows the frequency of adoptively transferred P25TCRTh1 (top row) or endogenous (bottom row) CD4⁺ T cells producing IFN-γ at various time points after intravenous treatment with synthetic Ag85B peptide 25. Flow cytometry dot plots show lung CD4⁺ cells from a representative mouse at the indicated time point after treatment with peptide 25. Values indicate the proportion of IFN-γ′ cells among the CD45.2⁺ or CD45.2⁺, CD4⁺ population for each mouse. Data shown are representative of n≧4 mice per group. FIG. 6B is a graph showing bacterial burden in the lungs of wild-type mice treated from day 28 to day 45 post-infection with intravenous Ag85B peptide 25 or OVA peptide control. Data points indicate the final bacterial population size for individual mice in each group after treatment with either peptide. Data shown are representative of n≧4 mice per group.

FIGS. 7A-B are schematic diagrams of CD4⁺ effector T cell activation at the site of M. tuberculosis infection. FIG. 7A shows that during the chronic stage of infection, Ag85B-specific CD4⁺ effector cells are activated at low frequencies, at least partially due to low bacterial expression of the antigen gene; bacteria are able to persist due to the low frequency of effector cell activation. FIG. 7B shows that administration of epitope peptide occupies previously-empty MHC class II and/or displaces previously-bound peptides and provides antigen for recognition by pre-existing epitope-specific CD4⁺ effector cells, resulting in their activation and consequent reduction of the lung bacterial burden.

FIG. 8 shows that brefeldin A treatment does not improve detection of IFN-γ produced by CD4⁺ cells in vivo. Frequency of lung CD4⁺ T cells on day 28 post-infection that stain with anti-IFN-γ antibody or isotype control is shown. Mice infected with M. tuberculosis were treated with 250 mg intravenous brefeldin A or left untreated. 6 hours after treatment, lungs were processed on ice in buffer alone or in buffer containing brefeldin A (20 mg/mL). Flow cytometry plots show lung CD4⁺ cells from a representative mouse in two experiments with n=3 mice. Values indicate the proportion of IFN-γ⁺ cells among CD4⁺ population for each mouse.

FIGS. 9A-B show that adoptively transferred P25TCRTh1 cells efficiently enter lung parenchyma and produce IFN-γ. FIG. 9A shows the percentage of P25TCRTh1 cells adoptively transferred into infected mice that stain PerCP⁻ or PerCP⁺ after intravenous treatment with PerCP labeled anti-CD4. Histogram gates indicate the fraction of CD4⁺, CD45.2⁺ lung cells that are either PerCP⁻ (parenchymal) or PerCP⁺ (intravascular). FIG. 9B shows the fraction of P25TCRTh1 cells from parenchymal (left, PerCP⁻) or intravascular (right, PerCP⁺) compartments that are activated in vivo to produce IFN-γ.

FIGS. 10A-B show that ablation of endogenous CD4⁺ T cells does not affect IFN-γ production by adoptively transferred P25TCRTh1 cells. FIG. 10A shows the frequency of (CD45.2⁺) endogenous CD4⁺ T cells among total lung cells from CD4-DTR mice 28 days after infection. Mice left untreated or were treated daily for 7 days prior to analysis with diphtheria toxin to ablate endogenous CD4⁺ T cells. FIG. 10B shows the effect of endogenous CD4⁺ T cell ablation on the fraction of (CD45.1⁺) P25TCRTh1 cells adoptively transferred on day 25 post-infection that are activated in the lungs to produce IFN-γ.

### DETAILED DESCRIPTION OF THE INVENTION

One aspect of the present invention relates to a method of treating a subject for an infectious disease. This method involves selecting a subject infected with an infectious disease-causing agent. The infectious disease-causing agent elicits, in the selected subject, an immune response that is insufficient to cure the selected subject of the infectious disease. The method also involves administering to the selected subject a synthetic peptide representing an antigen epitope associated with an immune response to the infectious agent. The immune response to the synthetic peptide is sufficient to treat the selected subject for the infectious disease.

According to one embodiment, infectious diseases treatable by the method of the present invention are chronic infectious diseases. Chronic infectious diseases amenable to treatment by the method of the present invention include both parasitic and fungal diseases. For example, diseases treatable by the method of the present invention include, without limitation, Histoplasmosis, Coccidioidomycosis, Cryptococcosis, Blastomycosis, typhoid fever (caused by Salmonella typhi or Salmonella paratyphi), Leishmaniasis and toxoplasmosis. In a preferred embodiment, the infectious disease is tuberculosis.

In carrying out the method of the present invention, a subject infected with an infectious disease is selected. Suitable subjects include, without limitation, any mammal, including, for example, a cow, horse, pig, sheep, goat, dog, cat, rabbit, rodent, non-human primate, or, in a preferred embodiment, a human. The subject may have a normal immune system. The subject may also be infected with a multi-drug resistant and/or extensively drug-resistant strain of the infectious disease-causing agent being treated by the method of the present invention.

The method of the present invention involves administering to the selected subject a synthetic peptide representing an antigen epitope associated with an immune response to the infectious agent. Administering the synthetic peptide to the selected subject can be carried out orally, intradermally, intramuscularly, intraperitoneally, intravenously, subcutaneously, or intranasally or otherwise by inhalation. The synthetic peptide may be administered alone or with suitable pharmaceutical carriers, and can be in solid or liquid form, such as tablets, capsules, powders, solutions, suspensions, or emulsions.

The synthetic peptide may be orally administered, for example, with an inert diluent, or with an edible carrier, or it may be enclosed in hard or soft shell capsules, or it may be compressed into tablets, or it may be incorporated directly with the food of the diet. For oral therapeutic administration, the synthetic peptide may be incorporated with excipients and used in the form of tablets, capsules, elixirs, suspensions, syrups, and the like. Such compositions and preparations should contain at least 0.1% of the synthetic peptide. The percentage of the synthetic peptide in these compositions may, of course, be varied and may conveniently be between about 2% to about 60% of the weight of the unit. The amount of the synthetic peptide of the present invention in such therapeutically useful compositions is such that a suitable dosage will be obtained.

The tablets, capsules, and the like may also contain a binder such as gum tragacanth, acacia, corn starch, or gelatin; excipients such as dicalcium phosphate; a disintegrating agent such as corn starch, potato starch, or alginic acid; a lubricant such as magnesium stearate; and a sweetening agent such as sucrose, lactose, or saccharin. When the dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

The synthetic peptide may also be administered parenterally. Solutions or suspensions of the synthetic peptide can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The synthetic peptide may also be administered directly to the airways in the form of an aerosol. For use as aerosols, the synthetic peptide in solution or suspension may be packaged in a pressurized aerosol container together with suitable propellants, for example, hydrocarbon propellants like propane, butane, or isobutane with conventional adjuvants. The synthetic peptide of the present invention also may be administered in a non-pressurized form such as in a nebulizer or atomizer.

According to one embodiment of the present invention, administering is carried out systemically in the selected subject.

Administering the synthetic peptide to the selected subject to treat the selected subject for the infectious disease may involve administration of the synthetic peptide twice-daily, daily, every 1-2 days, every 2-3 days, or weekly for a period of time sufficient to treat the subject. For example, administration may be carried out for 1-2 weeks, 3 weeks, 4 weeks, 1 month, 2 months, 3 months, or longer.

As used herein, treating a subject of an infectious disease includes reducing the extent of the infection. For example, the infection may be reduced in a subject by at least about 5%, 10%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 45%, 50%, 60%, 70%, 80%, 90%, or more. In one embodiment, the infection may no longer be detected in the subject after treatment. Thus, treating in the method of the present invention means a subject has a certain amount of infection and, following administration of the peptide, the amount of the infection in the subject is lower than it was prior to treatment.

In one embodiment, administering the synthetic peptide to the selected subject pursuant to the method of the present invention elicits an immune response that kills the infectious disease-causing agent. According to this embodiment, the infectious disease-causing agent may be a bacterium.

In one embodiment of the method of the present invention, the antigen epitope associated with an immune response to the infectious agent is bound to an MHC protein on the surface of an antigen-presenting cell. According to this embodiment, T cells in the selected subject's lungs are activated to treat tuberculosis. The antigen epitope may be an epitope of either an immunodominant antigen or a subdominant antigen.

According to one embodiment, the response to administering the synthetic peptide is activation of CD4⁺ and/or CD8⁺ T cells.

Synthetic peptides of the present invention have an antigen epitope associated with an immune response to the infectious agent being treated by the method of the present invention.

Synthetic peptides of the present invention can be made by well known recombinant techniques. For example, a nucleic acid molecule encoding a suitable synthetic peptide can be introduced into an expression system or vector of choice using conventional recombinant technology. Generally, this involves inserting the nucleic acid molecule into an expression system to which the molecule is heterologous (i.e., not normally present). The heterologous nucleic acid molecule is inserted into the expression system or vector in proper sense (5′→3′) orientation and correct reading frame. The vector contains the necessary elements for the transcription and translation of the inserted protein-coding sequences.

U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture.

Recombinant genes may also be introduced into viruses, such as vaccinia virus. Recombinant viruses can be generated by transfection of plasmids into cells infected with virus.

Suitable vectors include, but are not limited to, the following viral vectors: lambda vector system gt11, gt WES.tB, Charon 4, and plasmid vectors such as pBR322, pBR325, pACYC177, pACYC184, pUC8, pUC9, pUC18, pUC19, pLG339, pR290, pKC37, pKC101, SV 40, pBluescript II SK+/− or KS+/− (see “Stratagene Cloning Systems” Catalog (1993) from Stratagene, La Jolla, Calif., which is hereby incorporated by reference in its entirety), pQE, pIH821, pGEX, pET series (see F. W. Studier et. al., “Use of T7 RNA Polymerase to Direct Expression of Cloned Genes,” Gene Expression Technology Vol. 185 (1990), which is hereby incorporated by reference in its entirety), and any derivatives thereof. Recombinant molecules can be introduced into cells via transformation, particularly transduction, conjugation, mobilization, or electroporation. The DNA sequences are cloned into the vector using standard cloning procedures in the art, as described by Sambrook et al., Molecular Cloning: A Laboratory Manual, Cold Springs Laboratory, Cold Springs Harbor, N.Y. (1989), which is hereby incorporated by reference in its entirety.

A variety of host-vector systems may be utilized to express the protein-encoding sequence of the present invention. Primarily, the vector system must be compatible with the host cell used. Host-vector systems include but are not limited to the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria. The expression elements of these vectors vary in their strength and specificities. Depending upon the host-vector system utilized, any one of a number of suitable transcription and translation elements can be used.

Different genetic signals and processing events control many levels of gene expression (e.g., DNA transcription and messenger RNA (“mRNA”) translation).

Transcription of DNA is dependent upon the presence of a promoter which is a DNA sequence that directs the binding of RNA polymerase and thereby promotes mRNA synthesis. The DNA sequences of eukaryotic promoters differ from those of prokaryotic promoters. Furthermore, eukaryotic promoters and accompanying genetic signals may not be recognized in or may not function in a prokaryotic system, and, further, prokaryotic promoters are not recognized and do not function in eukaryotic cells.

Similarly, translation of mRNA in prokaryotes depends upon the presence of the proper prokaryotic signals which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression, see Roberts and Lauer, Methods in Enzymology 68:473 (1979), which is hereby incorporated by reference in its entirety.

Promoters vary in their “strength” (i.e., their ability to promote transcription). For the purposes of expressing a cloned gene, it is desirable to use strong promoters in order to obtain a high level of transcription and, hence, expression of the gene. Depending upon the host cell system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited to, lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Bacterial host cell strains and expression vectors may be chosen which inhibit the action of the promoter unless specifically induced. In certain operons, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Specific initiation signals are also required for efficient gene transcription and translation in prokaryotic cells. These transcription and translation initiation signals may vary in “strength” as measured by the quantity of gene specific messenger RNA and protein synthesized, respectively. The DNA expression vector, which contains a promoter, may also contain any combination of various “strong” transcription and/or translation initiation signals. For instance, efficient translation in E. coli requires a Shine-Dalgarno sequence about 7-9 bases 5′ to the initiation codon (ATG) to provide a ribosome binding site. Thus, any SD-ATG combination that can be utilized by host cell ribosomes may be employed. Such combinations include but are not limited to the SD-ATG combination from the cro gene or the N gene of coliphage lambda, or from the E. coli tryptophan E, D, C, B or A genes. Additionally, any SD-ATG combination produced by recombinant DNA or other techniques involving incorporation of synthetic nucleotides may be used.

Depending on the vector system and host utilized, any number of suitable transcription and/or translation elements, including constitutive, inducible, and repressible promoters, as well as minimal 5′ promoter elements may be used.

A nucleic acid molecule encoding a synthetic peptide suitable for use in the method of the present invention, a promoter molecule of choice, a suitable 3′ regulatory region and, if desired, a reporter gene, may be incorporated into a vector-expression system of choice to prepare a nucleic acid construct using standard cloning procedures known in the art, such as described by Sambrook et al., Molecular Cloning: A Laboratory Manual, Third Edition, Cold Spring Harbor: Cold Spring Harbor Laboratory Press, New York (2001), which is hereby incorporated by reference in its entirety.

The nucleic acid molecule encoding the synthetic peptide of choice is inserted into a vector in the sense (i.e., 5′→3′) direction, such that the open reading frame is properly oriented for the expression of the encoded protein under the control of a promoter of choice. Single or multiple nucleic acids may be ligated into an appropriate vector in this way, under the control of a suitable promoter, to prepare a suitable nucleic acid construct.

Once the isolated nucleic acid molecule encoding the synthetic peptide has been cloned into an expression system, it is ready to be incorporated into a host cell. Recombinant molecules can be introduced into cells via transformation, particularly transduction, conjugation, lipofection, protoplast fusion, mobilization, particle bombardment, or electroporation. The DNA sequences are cloned into the host cell using standard cloning procedures known in the art, as described by Sambrook et al., Molecular Cloning: A Laboratory Manual, Second Edition, Cold Springs Laboratory, Cold Springs Harbor, N.Y. (1989), which is hereby incorporated by reference in its entirety. Suitable hosts include, but are not limited to, bacteria, virus, yeast, fungi, mammalian cells, insect cells, plant cells, and the like.

Alternatively, synthetic peptides can be synthesized from individual amino acids. In particular, the synthetic peptides may be synthesized from monomeric amino acids through condensation reactions that form peptide bonds. The proteins are constructed sequentially by coupling the C-terminus of a monomeric amino acid with the N-terminus of the growing peptide. To prevent unwanted reactions involving the amino groups and carboxyl groups of the side chains during the coupling steps, a protecting (i.e., blocking) group is attached to the side chains. In the last steps of synthesis, the side chain-protecting groups are removed and the peptide is cleaved from the resin on which synthesis occurs.

A typical task in the design and synthesis of peptides is to identify a linear sequence of amino acid residues exhibiting a desired biological activity, e.g., representing an antigen epitope associated with an immune response to the infectious agent to be treated in the subject. In the simplest case, there are only two parameters subject to optimization: (i) the number of sequence positions (peptide length), and (ii) the type of residue present at each sequence position.

The typical experimental approach to peptide design is to perform large-scale random screening which has become feasible due to recent advances in peptide synthesis and activity detection techniques (Gausepohl et al., “Automated Multiple Peptide Synthesis,” Pept. Res. 5:315-320 (1992); Kramer and Schneider-Mergener, “Synthesis and Screening of Peptide Libraries on Cellulose Membrane Supports,” Methods Mol. Biol. 87:25-39 (1998); Kramer et al., “Molecular Basis for the Binding Promiscuity of an Anti-p24 (HIV-1) Monoclonal Antibody,” Cell 91:799-809 (1997); which are hereby incorporated by reference in their entirety). Blind screening is essential if no information about function determining residue patterns is available. This is particularly true when conventional structure-based molecule modeling cannot be performed due to the lack of high-resolution receptor structures. If however, a template peptide or other information is already known that can be used to limit the search space, it is worthwhile following some kind of rational design (Schneider et al., “Peptide Design by Artificial Neural Networks and Computer-based Evolutionary Search,” Proc. Natl. Acad. Sci. (USA) 95:12179-12184 (1998); Schneider and Wrede, “The Rational Design of Amino Acid Sequences by Artificial Neural Networks and Simulated Molecular Evolution: De novo Design of an Idealized Leader Peptidase Cleavage Site,” Biophys. J. 66:335-344 (1994); Huang et al., “Development of a Common 3D Pharmacophore for Delta-opioid Recognition from Peptides and Non-peptides Using a Novel Computer Program,” J. Comput. Aided Mol. Des. 11:21-28 (1997); Mee et al., “Design of Active Analogues of a 15-residue Peptide Using D-optimal Design, QSARand a Combinatorial Search Algorithm,” J. Pept. Res. 49:89-102 (1997); which are hereby incorporated by reference in their entirety).

In one embodiment of the present invention, the synthetic peptide is peptide 25 of SEQ ID NO:10.

Another aspect of the present invention is directed to a peptide comprising an antigen epitope associated with an immune response to Mycobacterium tuberculosis. The peptide elicits an immune response in a subject that reduces the extent of infection in the subject beyond what a normal immune response in the subject can reduce.

Peptides of the present invention may elicit an immune response in a subject that reduces the extent of infection (e.g., bacterial or fungal burden) in a subject by at least about 5%, 10%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 45%, 50%, 60%, 70%, 80%, 90%, or more beyond what the subject's normal immune response can control.

### EXAMPLES

The following examples are provided to illustrate embodiments of the present invention but are by no means intended to limit its scope.

### Example 1

### Materials and Methods

Mice

C57BL/6, B6.5JL-Ptprc^{a} Pepc^{b}/BoyJ (CD45.1⁺), and MHCII KO mice for aerosol M. tuberculosis infection experiments were either bred in the New York University School of Medicine Skirball animal facility or purchased from Taconic Farms, Inc. P25TCR-Tg mice, whose CD4⁺ T cells express a transgenic T-cell antigen receptor that recognizes the complex of peptide 25 (aa 240-254) of M. tuberculosis Ag85B and the mouse MHC II allele I-A^{b} were prepared on a C57BL/6 background, as previously described (Tamura et al., “The Role of Antigenic Peptide in CD4⁺ T Helper Phenotype Development in a T Cell Receptor Transgenic Model,” Int. Immunol. 16:1691-9 (2004); Wolf et al., “Initiation of the Adaptive Immune Response to Mycobacterium tuberculosis Depends on Antigen Production in the Local Lymph Node, Not the Lungs,” J. Exp. Med. 205:105-15 (2008), which are hereby incorporated by reference in their entirety). All animal experiments were done in accordance with procedures approved by the NYU School of Medicine Institutional Animal Care and Use Committee and in strict accordance with the recommendations in the Guide for the Care and Use of Laboratory Animals of the National Institutes of Health under the Assurance of Compliance Number A3435-01.

M. tuberculosis In Vitro Growth and Aerosol Mouse Infection

Wild-type M. tuberculosis H37Rv was originally obtained from ATCC. Frozen stocks for aerosol infection and in vitro use were prepared and stored at −80° C. GFP-expressing H37Rv and Ag85B null (ΔAg85B) strains of M. tuberculosis were generated as previously described (Wolf et al., “Initiation of the Adaptive Immune Response to Mycobacterium tuberculosis Depends on Antigen Production in the Local Lymph Node, Not the Lungs,” J. Exp. Med. 205:105-15 (2008); Wolf et al., “Mycobacterium tuberculosis Infects Dendritic Cells with High Frequency and Impairs their Function In Vivo,” J. Immunol. 179:2509-19 (2007), which are hereby incorporated by reference in their entirety). M. tuberculosis cultures were grown in 10 mL Middlebrook 7H9 liquid medium supplemented with 10% v/v albumin dextrose catalase enrichment and incubated under shaking conditions at 37° C. Mice at 8-12 weeks of age were infected with ˜100 CFU of M. tuberculosis via the aerosol route using an Inhalation Exposure Unit (Glas-Col) as previously described (Wolf et al., “Mycobacterium tuberculosis Infects Dendritic Cells with High Frequency and Impairs their Function In Vivo,” J. Immunol. 179:2509-19 (2007), which is hereby incorporated by reference in its entirety). To verify inoculum size, 3-5 infected mice were euthanized 24 hours after infection and lungs were homogenized and plated on Middlebrook 7H11 medium supplemented with 10% v/v albumin dextrose catalase enrichment. To determine bacterial population size at time points post-infection, lungs were homogenized, diluted in PBS+Tween-80 (0.5%), and added to 7H11 plates. Plates were incubated at 37° C. for 3 weeks and single colonies were counted. To determine M. tuberculosis survival in stationary culture, 7H9 medium was inoculated with H37Rv or CPE85B, grown in shaking conditions to saturation (O.D.₆₀₀>1.0), and initial CFUs were measured. Cultures were then placed in a stationary incubator at 37° C. for 17 days, and final CFUs were measured.

Fluorescent Microscopy of Frozen Tissue Sections

C57BL/6 mice were infected with M. tuberculosis H37Rv and on day 25 post-infection received 1×10⁶ CFP⁺ P25TCRTh1 cells via adoptive transfer. On day 28 post-infection, lungs were perfused and frozen in OCT before 5 μm sectioning and fixation in cold acetone. Sections were stained with DAPI to label nuclei and analyzed on a Leica DMRB fluorescent microscope (objective: Leica PL Fluotar 20×/0.50) equipped with a Spot RT digital camera. Separate images for DAPI and CFP fluorescence were acquired and merged using Spot software.

P25TCRTh1 CD4⁺ Effector T Cells

P25 TCR-Tg CD4⁺ Th1 effector cells were generated in vitro as follows: naïve CD4⁺ T cells were magnetically isolated from lymph node cell suspensions of P25 TCR-Tg mice (or for fluorescent microscopy, a P25TCR-Tg mouse expressing CFT under control of the ubiquitin promoter) using CD4 (L3T4) microbeads and an AutoMACS (Miltenyi Biotech). P25TCR-Tg CD4⁺ T cells were co-cultured with irradiated C57BL/6 splenocytes in the presence of mouse IL-12p70 (10 ng/ml), mouse IL-2 (5 ng/ml), anti-IL-4 neutralizing antibody (50 ng/ml), and synthetic peptide 25 (0.5 μM). Cells were cultured at 37° C. with 5% CO₂. On days 3 and 5 of culture, cells were split 1:3 with fresh media containing IL-12p70, IL-2, and anti-IL-4, but no peptide 25. Cells were washed with PBS and counted on day 7 of culture before use for in vitro or in vivo assays. For in vitro restimulation, P25TCRTh1 cells were co-cultured with irradiated C57BL/6 splenocytes for 24 hours in RPMI-10 in the presence or absence of peptide 25 (0.5 μM) or bone marrow derived dendritic cells infected with M. tuberculosis (MOI: 0.1). Cells were collected and analyzed by flow cytometry for intracellular IFN-γ, or culture supernatants were analyzed for IFN-γ by ELISA. For in vivo experiments, 1×10⁶ P25TCRTh1 cells were injected via tail vein or retro-orbital sinus into recipient mice at various time points post-infection. Cells were isolated from lungs of recipient mice 72 hours after adoptive transfer and analyzed by flow cytometry.

Naïve P25TCR-Tg T Cell Proliferation

3×10⁶ CFSE-labeled CD4⁺ T cells, harvested from the lymph nodes of P25TCR-Tg mice, were adoptively transferred into infected recipients at various time points post-infection. 7 days after adoptive transfer, mediastinal lymph nodes were harvested from recipient mice and cells were analyzed for CFSE dilution by flow cytometry.

Generation of CPE85B Strain of M. tuberculosis

The Ag85B null strain of M. tuberculosis (ΔAg85B), previously created from wild-type H37Rv (Wolf et al., “Initiation of the Adaptive Immune Response to Mycobacterium tuberculosis Depends on Antigen Production in the Local Lymph Node, Not the Lungs,” J. Exp. Med. 205:105-15 (2008), which is hereby incorporated by reference in its entirety), was used as a background strain for generating CPE85B. Both the hspX promoter sequence, consisting of 254 bp directly 5′ of the hspX start codon, as well as the fbpB open reading frame were amplified by PCR from H37Rv genomic DNA. Each of these fragments was ligated into the pMV306 integrating vector to create a recombinant construct, whose sequence was verified by Sanger sequencing performed by the NYU DNA sequencing facility. ΔAg85B was grown in 7H9 liquid media and transformed with this construct via electroporation. The reaction was plated on 7H11 plates containing 25 μg/ml kanamycin to select for bacteria incorporating the construct into the M. tuberculosis chromosome. Presence of the construct in kanamycin resistant colonies was verified by PCR. Expression and secretion of Ag85B by CPE85B was confirmed by SDS-PAGE and anti-Ag85B western blot of supernatants from 7H9 liquid medium after stationary culture. For stationary culture-induced expression of Ag85B by the CPE85B strain, 10 mL cultures were grown to late phase (OD₆₀₀˜1.0) in normal shaking conditions, then flasks were sealed and transferred to a stationary incubator for >1 week before supernatants were collected.

RT-qPCR of Bacterial mRNA from Infected Mouse Lungs

To quantitate expression of M. tuberculosis genes during mouse infection, lungs of infected mice were rapidly placed into a solution of RNAlater (Ambion) and stored overnight at room temperature in accordance with manufacturer recommendations to allow permeation of the tissue. Thereafter, samples for RNA isolation were stored at −80° C. When comparing expression of genes at various time points, tissues were transferred to TRIzol (Invitrogen) and quickly homogenized using a Tissue Tearor™ homogenizer to disrupt mouse cells. Lung homogenates were centrifuged to pellet intact bacterial cells, and supernatants discarded. M. tuberculosis pellets were disrupted with zirconia/silica beads, RNA was extracted, and RT-qPCR was carried out as previously described (Banaiee et al., “Regulation of Mycobacterium tuberculosis whiB3 in the Mouse Lung and Macrophages,” Infect. Immun. 74:6449-57 (2006), which is hereby incorporated by reference in its entirety). The following RT-qPCR primers were used: 16S rRNA: RT 5-ATTACGTGCTGGCAACATGA-3 (SEQ ID NO:1), qPCR For 5-GCCGTAAACGGTGGGTACTA-3 (SEQ ID NO:2), qPCR Rev 5-TGCATGTCAAAC-CCAGGTAA-3 (SEQ ID NO:3); hspx/acr/Rv2031c: RT 5-GAATGCCCTTGTCGTAG-GTG-3 (SEQ ID NO:4), qPCR For 5-AGATGAAAGAGGGGCGCTAC-3 (SEQ ID NO:5), qPCR Rev 5-TAATGTCGTCCTCGTCAGCA-3 (SEQ ID NO:6); fbpB/Rv1886c: RT 5-TCCTGGAACTTCAGGTTGCT-3 (SEQ ID NO:7), qPCR For 5-ACCCCCAGC-AGTTCATCTAC-3 (SEQ ID NO:8), qPCR Rev 5-TTCCCGCAATAAACCCATAG-3 (SEQ ID NO:9).

Tissue Processing and Flow Cytometry

To isolate cells from infected tissues for flow cytometry, mice were euthanized with CO₂ followed by cervical dislocation. Tissues were removed and mechanically disrupted by mincing in RPMI as previously described (Wolf et al., “Mycobacterium tuberculosis Infects Dendritic Cells with High Frequency and Impairs their Function In Vivo,” J. Immunol. 179:2509-19 (2007), which is hereby incorporated by reference in its entirety) or using a gentleMACST™ dissociator (Miltenyi Biotec) in the manufacturer-recommended HEPES buffer. Lung suspensions were incubated in Collagenase D and DNase at 37° C. with 5% CO₂ for 30 minutes and cells were isolated by forcing suspensions through a 70 μM cell strainer. RBCs were removed by ACK lysis and live cells counted by trypan blue exclusion. Cell suspensions were stained using the following fluorescently-labeled antibodies (Biolegend, BD Pharmingen, or eBioscience): anti-CD3 PE, anti-CD4 (L3T4) FITC, anti-CD45.2 PerCP, anti-CD45.1 Pacific Blue, anti-IFN-γ (XMG1.2) APC, and rat IgG1 APC isotype control. Flow cytometry was performed using a FACSCalibur or LSR II (BD Biosciences) at the NYU Cancer Institute Flow Cytometry and Cell Sorting facility. Analysis of flow cytometry data was performed using FlowJo software.

Detection of IFN-γ Producing Cells by Direct Intracellular Cytokine Staining

To detect intracellular IFN-γ produced by cells in vivo, a protocol was developed based on a previous study (Liu & Whitton, “Cutting Edge: Re-Evaluating the In Vivo Cytokine Responses of CD8⁺ T Cells During Primary and Secondary Viral Infections,” J. Immunol. 174:5936-40 (2005), which is hereby incorporated by reference in its entirety). In contrast to this study, however, optimal detection of IFN-γ producing cells from the lungs of mice infected with M. tuberculosis did not require treatment of mice with i.v. brefeldin A or inclusion of brefeldin A in tissue processing buffers. Instead, after euthanasia, tissues were rapidly placed on ice and all cell isolation steps except collagenase/DNase digestion (37° C. for 30 minutes) and ACK lysis (room temperature for 5 minutes) were carried out quickly and on ice. Cells were stained for surface markers at 4° C. for 30 minutes, followed by permeabilization and fixation with Cytofix/Cytoperm (BD Biosciences) at 4° C. for 20 minutes. Finally, fixed cells were stained with anti-IFN-γ or a rat IgG1 isotype control at 4° C. for 30 minutes. Flow cytometry dot plot gates for IFN-γ′ cells were set based on comparison with isotype control and unpermeabilized cells stained for IFN-γ.

CD4⁺ T Cell Depletion

Mice were treated with an intra-peritoneal dose of 500 μg of either monoclonal antibody GK1.5, which depletes CD4⁺ T cells, or a rat IgG2b isotype control (LTF-2) every 6 days from day 28 to day 50 post-infection. Efficiency of CD4⁺ T cell depletion 6 days after an antibody treatment was determined to be >95% by flow cytometry of cell suspensions from lungs, spleen, and blood. In mice treated with LTF-2 isotype control, no differences were observed in CD4⁺ T cell number or bacterial burden when compared to untreated mice.

Selective Ablation of Endogenous CD4⁺ T Cells

To determine the influence of endogenous CD4⁺ T cells on the response of adoptively transferred P25TCRTh1 cells in vivo, a system was developed to deplete endogenous CD4⁺ T cells selectively from infected mice. Mice expressing Cre recombinase under control of the CD4 promoter were crossed with those carrying an inducible Diphtheria Toxin Receptor (iDTR) allele, whose baseline expression is prevented by a stop codon flanked by loxp sites (Buch et al., “A Cre-Inducible Diphthera Toxin Receptor Mediates Cell Lineage Ablation After Toxin Administration,” Nat. Methods 2:419-426 (2005), which is hereby incorporated by reference in its entirety). Progeny of this cross (CD4-DTR) carry CD4⁺ T cells that are sensitive to Diphtheria Toxin mediated ablation. CD4-DTR mice were infected with H37Rv and received daily intraperitoneal doses of DT (100 ng) to ablate endogenous CD4⁺ T cells from day 21 to day 28 post-infection. The efficiency of CD4⁺ T cell ablation in the lungs was determined by flow cytometry to be 48.9%. P25TCRTh1 cells were adoptively transferred on day 25 post-infection and the frequency of IFN-γ production was assessed on day 28 post-infection.

Assessment of Intravascular and Extravascular Location of Adoptively-Transferred P25TCRTh1 Cells

On day 25 post-infection, wild-type mice infected with M. tuberculosis H37Rv received P25TCRTh1 cells via adoptive transfer. On day 28 post-infection, mice were treated intravenously with 800 ng (at 4.0 ng/mL) PerCP-labeled anti-CD4 (RM4-5). Fifteen minutes later, mice were euthanized and total lung cells were stained with FITC-labeled anti-CD4 (GK1.5). Lung cells stained by anti-CD4-PerCP were considered to be CD4⁺ T cells residing in the intravascular compartment at the time of antibody injection. Cells staining positive for anti-CD4-FITC and negative for PerCP were considered to be CD4⁺ T cells residing in an extravascular or parenchymal lung compartment protected from labeling with intravenous antibody. IFN-γ production in vivo was assessed by intracellular staining of all cells with APC-labeled anti-IFN-γ as previously described.

Systemic Treatment of Mice with Synthetic Peptides

Mice were intravenously treated with 100 μg of Ag85B peptide 25 (FQDAYNAAGGHNAVF (SEQ ID NO:10)) or OVA peptide control (ISQAVHAAHAEINEAGR (SEQ ID NO:11)) in 100 μl sterile PBS via tail vein or retro-orbital sinus. Peptides were synthesized by EZBiolab or Peptides International to a purity of >95%.

### Statistical Analyses

Data shown are representative of 2 or more experimental replicates. In all figures, error bars indicate mean±SEM. To determine statistical significance when comparing experimental values from two groups of mice, one- or two-tailed student's t-tests were routinely used, each where appropriate. To compare the growth rate of H37Rv and CPE85B in vivo, a non-linear regression analysis (curve fit) with F-test was used to determine whether a single curve could account for both data sets. In mouse survival experiments, Logrank test was used to evaluate statistical significance when comparing survival of one mouse strain after infection with either of the two bacterial strains. *=p<0.05; **=p<0.005; n.s.=not significant.

### Example 2

### Prevalence of CD4⁺ T Cells Activated to Produce IFN-γ in the Lungs of M. tuberculosis-Infected Mice

It was hypothesized that M. tuberculosis evades adaptive immunity by modulating the activation of CD4⁺ effector T cells at the site of infection in the lungs.

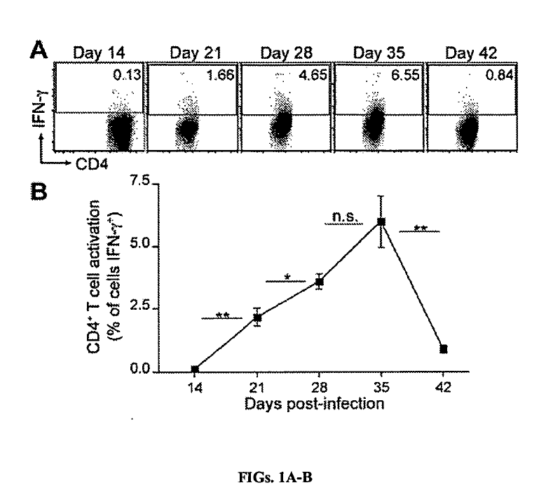

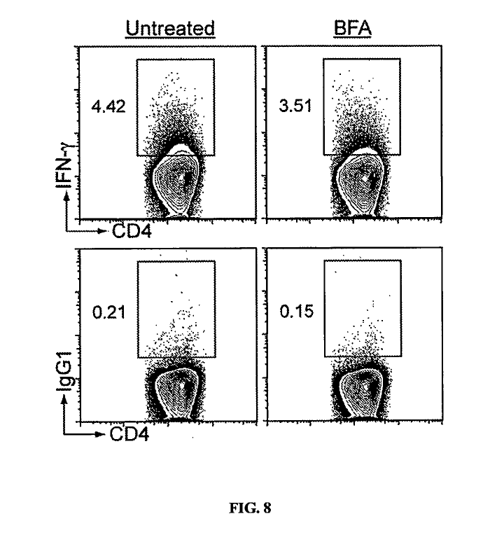

Since in vitro studies have revealed evidence that M. tuberculosis modulates MHC class II antigen presentation (Harding et al., “Regulation of Antigen Presentation by Mycobacterium tuberculosis: A Role for Toll-Like Receptors,” Nat. Rev. Microbiol. 8:296-307 (2010); Noss et al., “Toll-Like Receptor 2-Dependent Inhibition of Macrophage Class II MHC Expression and Antigen Processing by 19-kDa Lipoprotein of Mycobacterium tuberculosis,” J. Immunol. 167:910-918 (2001); Singh et al., “Processing and Presentation of a Mycobacterial Antigen 85B Epitope by Murine Macrophages is Dependent on the Phagosomal Acquisition of Vacuolar Proton ATPase and in situ Activation of Cathepsin D,” J. Immunol. 177:3250-3259 (2006); Hmama et al., “Attenuation of HLA-DR Expression by Mononuclear Phagocytes Infected with Mycobacterium tuberculosis is Related to Intracellular Sequestration of Immature Class II Heterodimers,” J. Immunol. 161:4882-4893 (1998); Pancholi et al., “Sequestration from Immune CD4+ T Cells of Mycobacteria Growing in Human Macrophages,” Science 260:984-986 (1993), which are hereby incorporated by reference in their entirety), the work reported here focused on in vivo activation of CD4⁺ T cells in the lungs. It was reasoned that, if M. tuberculosis-infected cells do not present antigens efficiently to effector T cells in the lungs, then the frequency of activation of effector functions of CD4⁺ cells would also be low at the site of infection. To test this, direct intracellular cytokine staining of lung cells from infected mice for IFN-γ was used, without ex vivo restimulation. It was found that that the frequency of IFN-γ expression by CD4⁺ T cells in the lungs varied with the time of infection (FIGS. 1A-B). IFN-γ⁺ CD4⁺ cells were undetectable in the lungs at day 14, increased in frequency beginning by day 21 to a peak at day 35 post-infection, and then markedly decreased afterward. No more than 7% of the bulk population of CD4⁺ T cells expressed IFN-γ at any time point after infection, and fewer than 1% expressed IFN-γ during the chronic phase. Other studies investigating IFN-γ production by CD8⁺ T cells in vivo have used treatment of mice with brefeldin A or inclusion of brefeldin A during cell isolation and staining (Hufford et al., “Antiviral CD8+ T Cell Effector Activities In Situ are Regulated by Target Cell Type,” J. Exp. Med. 208:167-180 (2010); Liu et al., “Cutting Edge: Re-Evaluating the In Vivo Cytokine Responses of CD8+ T Cells During Primary and Secondary Viral Infections,” J. Immunol. 174:5936-5940 (2005), which are hereby incorporated by reference in their entirety). However, it was determined that these methods did not improve detection of intracellular IFN-γ by CD4⁺ T cells during M. tuberculosis infection (FIG. 8). These data indicate that a small minority of polyclonal CD4⁺ T cells recruited to the lungs of M. tuberculosis-infected mice are activated to produce IFN-γ at a given time, and are consistent with defective antigen presentation, costimulation, and/or inhibition of effector T cell activation at the site of infection.

### Example 3

### Quantitating Antigen-Specific Effector Th1 Cell Responses in the Lungs of M. tuberculosis-Infected Mice

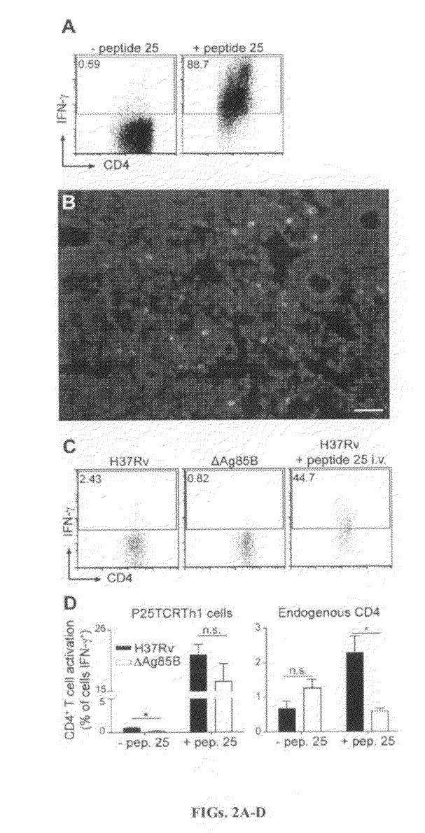

Since the low frequency of CD4⁺ T cell expression of IFN-γ in the lungs of M. tuberculosis-infected mice could be due to the presence of effector cells that traffic to the lungs but are not specific for M. tuberculosis antigens, the remainder of the studies described here were performed using CD4⁺ TCR transgenic T cells that specifically recognize a well-characterized immunodominant M. tuberculosis antigen. To quantitate the frequency of activation of M. tuberculosis antigen-specific effector cells in the lungs, CD4⁺ Th1 effector cells (P25TCRTh1 cells) were prepared from transgenic mice with a TCR specific for peptide 25 (amino acids 240-254) of Ag85B. When P25TCRTh1 cells were incubated with irradiated splenocytes in the absence of peptide 25, <1.0% of the cells expressed IFN-γ as detected by intracellular staining and flow cytometry, whereas addition of peptide 25 in vitro induced IFN-γ expression in ˜90% of cells (FIG. 2A). This result demonstrated that the frequency of IFN-γ staining in P25TCRTh1 cells can specifically assay antigen dependent stimulation of P25TCRTh1 cells.

### Example 4

### P25TCRTh1 Cells Recognize Antigen at Low Frequency In Vivo

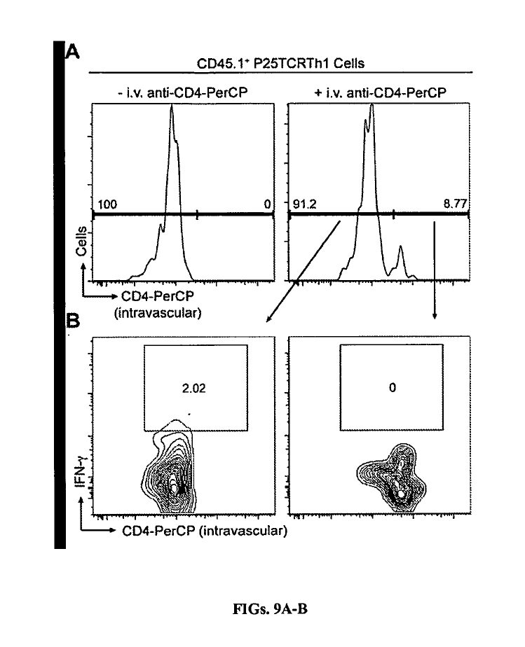

Since day 21 post-infection corresponds to an acute stage of infection when adaptive immune effector mechanisms have been initiated and reduce the rate of bacterial population growth in the lungs, and since it resembles the stage of LCMV infection in which a high frequency of antigen-specific CD8⁺ T cell responses are observed (Liu & Whitton, “Cutting Edge: Re-Evaluating the In Vivo Cytokine Responses of CD8+ T Cells During Primary and Secondary Viral Infections,” J. Immunol. 174:5936-5940 (2005), which is hereby incorporated by reference in its entirety), this time point was chosen for initial characterization of P25TCRTh1 cell responses in vivo. It was verified that adoptively transferred P25TCRTh1 cells traffic to the site of infection by examining sections of lungs from infected mice that had received CFP⁺ P25TCRTh1 cells. CFP⁺ cells were abundant in the lung parenchyma, and were concentrated in granulomas (FIG. 2B). Furthermore, it was determined that >85% of the transferred cells were protected from labeling by an i.v. injection of PerCP-labeled anti-CD4 antibody, indicating that adoptively transferred P25TCRTh1 cells efficiently migrate out of the lung vasculature into the parenchyma of infected lungs (FIG. 9A).

To determine the frequency of activation of antigen-specific CD4⁺ effector T cells in the lungs early in infection, P25TCRTh1 cells were adoptively transferred on day 18 and harvested on day 21 after infection of wild-type mice with wild-type M. tuberculosis H37Rv. The frequency of IFN-γ′ P25TCRTh1 cells isolated from the lungs was unexpectedly low at Day 21 post infection (FIGS. 2C and 2D). Approximately 1-2% of the transferred P25TCRTh1 cells were stimulated to produce IFN-γ in vivo at that time point (FIG. 2C), and this percentage was similar to the frequency of total endogenous lung CD4⁺ T cells expressing IFN-γ on day 21 post-infection (FIGS. 1B, 2D). Moreover, after intravenous injection of PerCP-labeled anti-CD4 antibody, the only IFN-γ′ P25TCRTh1 cells identified were PerCP negative (FIG. 9B), indicating that the responding cells were those that had migrated out of the vasculature into the lung parenchyma and were protected from staining by the in vivo injection of antibody.

It was verified that stimulation of P25TCRTh1 cells to express intracellular IFN-γ is due to recognition of Ag85B peptide 25 by transferring P25TCRTh1 cells into mice infected with an Ag85Bnull strain of M. tuberculosis (DAg85B), which is equivalent to wild-type H37Rv in virulence (Wolf et al., “Initiation of the Adaptive Immune Response to Mycobacterium tuberculosis Depends on Antigen Production in the Local Lymph Node, Not the Lungs,” J. Exp. Med. 205:105-115 (2008), which is hereby incorporated by reference in its entirety). A lower mean percentage (0.74%) of P25TCRTh1 cells isolated from DAg85B-infected mice expressed IFN-γ than those from H37Rv-infected mice (FIGS. 2C and 2D). This indicates that in vivo IFN-γ production by P25TCRTh1 cells is antigen-dependent and not the consequence of inflammatory cytokines present at the site of infection. Several alternative approaches to detecting effector T cell activation in the lungs were also evaluated. P25TCRTh1 cells expressed both CD25 and CD44 prior to adoptive transfer, which excluded their use in evaluating effector cell activation in vivo. Surface expression of CD69 was induced after adoptive transfer of P25TCRTh1 effector cells into H37Rvinfected mice. However, similar induction of CD69 in mice infected with DAg85B was found, indicating that it did not specifically reflect antigen-dependent effector cell activation. This result, together with evidence that CD69 can be induced by costimulation and by certain cytokines present at the site of M. tuberculosis infection (Abe et al., “Distinct Signal Transduction in Mouse CD4+ and CD8+ Splenic T Cells After CD28 Receptor Ligation,” J. Immunol. 154:985-997 (1995); Sun et al., “Type I Interferon-Mediated Stimulation of T Cells by CpG DNA,” J. Exp. Med. 188:2335-2342 (1998); Lahn et al., “Early Preferential Stimulation of Gamma Delta T Cells by TNF-Alpha,” J. Immunol. 160:5221-5230 (1998), which are hereby incorporated by reference in their entirety), indicates that expression of intracellular IFN-γ is the most accurate reporter of antigen specific Th1 effector cell activation in the lungs. Together, these results indicate that even though they traffic efficiently to the site of infection, Ag85B peptide 25-specific CD4⁺ effector cells are activated to execute their Th1 effector function at low frequency in the lungs of M. tuberculosis-infected mice.

### Example 5

### P25TCRTh1 Cells are Capable of Responding to Antigen at the Site of Infection

Although IFN-γ production by P25TCRTh1 cells at day 21 was dependent on Ag85B, the frequency of IFN-γ⁺ cells was surprisingly low in H37Rv infected mice. One possible explanation for the low frequency of activation of effector cells is that their cognate antigen is not available for recognition at the site of infection. To test this hypothesis, antigen was provided in vivo by injecting peptide 25 intravenously into mice that had been infected 21 days earlier. When P25TCRTh1 recipient, H37Rv-infected mice received peptide 25 six hours prior to lung cell harvest, the frequency of IFN-γ⁺ P25TCRTh1 cells increased to 20-50% (FIGS. 2C and 2D). Similarly, peptide 25 injection stimulated a higher frequency of IFN-γ expression by endogenous CD4⁺ T cells from mice infected with H37Rv (FIGS. 2C and 2D), consistent with prior evidence that peptide 25 of Ag85B is a dominant antigen in C57BL/6 mice infected with M. tuberculosis (Yanagisawa et al., “Mapping of V beta 11+Helper T Cell Epitopes on Mycobacterial Antigen in Mouse Primed with Mycobacterium tuberculosis,” Int. Immunol. 9:227-237 (1997); Jung et al., “Properties and Protective Value of the Secondary Versus Primary T Helper Type I Response to Airborne Mycobacterium tuberculosis Infection in Mice,” J. Exp. Med. 201:1915-1924 (2005), which are hereby incorporated by reference in their entirety). P25TCRTh1 cells transferred into DAg85B-infected recipients were also stimulated at a higher frequency after intravenous peptide 25 treatment, while endogenous CD4⁺ T cells from DAg85B-infected mice did not respond to peptide 25 with increased IFN-γ expression (FIG. 2D). The failure of endogenous CD4⁺ T cells from DAg85B-infected mice to respond to peptide 25 injection reflects the absence of Ag85B peptide 25-specific effector T cells generated in response to this infection. These results indicate that the frequency of IFN-γ⁺ P25TCRTh1 cells is an accurate and specific measure of CD4⁺ effector T cell stimulation in response to presentation of Ag85B peptide 25 in vivo. The observation that in vivo IFN-γ responses to peptide 25 injection depend on the presence of previously-generated (endogenous or transferred) peptide 25-specific effector T cells indicates that the responses are not due to a nonspecific effect of the epitope peptide on costimulation or responses of CD4⁺ T cells with specificity for other antigens. In addition, they demonstrate that if antigen is made available to them, adoptively transferred P25TCRTh1 cells can respond to antigen in the infected lungs, and they provide evidence against an exclusive role for T regulatory cells and/or suppressive cytokines in limiting the activation of CD4⁺ effector cells at the site of M. tuberculosis infection in the lungs.

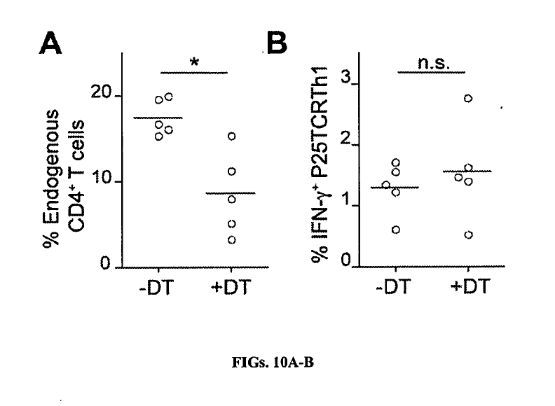

To further characterize the in vivo assay system, and to evaluate the possibility that low frequencies of P25TCRTh1 responses are attributable to either competition for antigen by endogenous CD4⁺ T cells and/or a dominant effect of T regulatory cells, endogenous T cells were specifically ablated from M. tuberculosis-infected CD4-DTR mice (Buch et al., “A Cre-Inducible Diphtheria Toxin Receptor Mediates Cell Lineage Ablation After Toxin Administration,” Nat. Methods 2:419-426 (2005), which is hereby incorporated by reference in its entirety) prior to assaying P25TCRTh1 responses in vivo. Compared to untreated mice, DT treatment reduced the fraction of endogenous CD4⁺ T cells in the lung by an average of 48.9%, p=0.0053 (FIG. 10A). However, this had no effect on the percentage of P25TCRTh1 cells activated to produce IFN-γ (FIG. 10B). These results strongly suggest that the low frequency of activation of P25TCRTh1 cells is caused neither by competition for peptide 25:MHC II complexes by endogenous CD4⁺ T cells, nor by the influence of T regulatory cells in the lungs. It was therefore concluded that the response of adoptively transferred P25TCRTh1 cells is an accurate reflection of MHC II presentation of Ag85B peptide 25 by lung APCs during infection.

### Example 6

### Dynamics of M. tuberculosis-Specific CD4⁺ Effector T Cell Responses During the Course of Infection

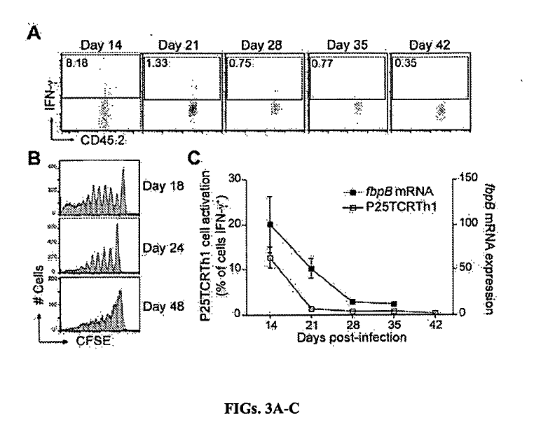

Adaptive immunity restricts progressive growth of M. tuberculosis, but it does not eliminate the bacteria from the lungs, which results in chronic infection in mice and latent infection in humans. To determine whether suboptimal activation of M. tuberculosis-specific T cells contributes to the ability of the bacteria to persist, it was first asked whether activation of P25TCRTh1 cells in the lungs changes as infection progresses to a chronic phase. To compare the frequency of effector T cell stimulation at various stages of infection, P25TCRTh1 cells were transferred into H37Rv-infected mice on day 11, 18, 25, 32, or 39 post-infection. Lung cells were harvested 72 hours after transfer (day 14, 21, 28, 35, or 42 post infection) and analyzed by flow cytometry for intracellular IFN-γ without ex vivo restimulation. The proportion of P25TCRTh1 cells producing IFN-γ was highest (−10%) on day 14 (FIGS. 3A and 3C). These results indicate that during the acute stage of infection, adoptively transferred P25TCRTh1 cells are stimulated in the lungs at a frequency comparable to that of TCR transgenic CD4⁺ effector cells at the site of injection of a protein antigen and adjuvant (McLachlan et al., “Dendritic Cell Antigen Presentation Drives Simultaneous Cytokine Production by Effector and Regulatory T Cells in Inflamed Skin,” Immunity 30:277-288 (2009), which is hereby incorporated by reference in its entirety). In contrast, expression of IFN-γ by endogenous (CD45.22⁻) CD4⁺ cells was rare (<0.1%) at that time point (FIGS. 1B and 1C). The difference between transferred and endogenous cell responses on day 14 is consistent with the previous observation that initiation of adaptive immunity to M. tuberculosis is delayed until day 11-14 post-infection and, consequently, endogenous CD4⁺ effector T cells specific for M. tuberculosis antigens are first detected in the lungs on day 17 post infection (Wolf et al., “Initiation of the Adaptive Immune Response to Mycobacterium tuberculosis Depends on Antigen Production in the Local Lymph Node, Not the Lungs,” J. Exp. Med. 205:105-115 (2008), which is hereby incorporated by reference in its entirety).

The frequency of IFN-γ production by P25TCRTh1 cells progressively decreased from day 14 to day 42 post-infection, indicating a decrease in the efficiency of peptide 25-specific T cell stimulation as infection enters its chronic phase (FIGS. 3A and 3C). These results with TCR transgenic CD4⁺ effector cells closely mimic the results observed with endogenous polyclonal CD4⁺ T cells after day 14 post-infection (FIGS. 1B and 1C). Although Ag85B peptide 25-specific responses reached an earlier peak and decreased earlier than did those of endogenous polyclonal CD4⁺ T cell responses, the results with the two cell populations were similar, with endogenous CD4⁺ effector T cell responses also diminishing by day 42 post-infection.

To determine whether activation of naïve Ag85B peptide 25-specific CD4⁺ T cells is also diminished in the later stages of M. tuberculosis infection, the response of adoptively transferred naïve P25 TCR-Tg T cells were assayed in the lung-draining mediastinal lymph nodes of H37Rv-infected mice at various time points post-infection. 7 days after transfer, lymph node cells were harvested and T cell proliferation was measured in vivo by flow cytometry using a CFSE dilution assay. The rate of naïve P25TCR-tg T cells was highest upon transfer into mice on day 18 post-infection, while fewer cells exhibited CFSE dilution at days 24 and 48 post-infection (FIG. 3B). These results indicate that decreased stimulation of P25TCRTh1 effector cells is also accompanied by decreased generation of peptide 25 specific effector T cells from naïve cells at later stages of infection.

### Example 7

### Progressive Decreases of P25TCRTh1 Cell Activation Accompany Decreased fbpB Expression

Since treatment of infected mice with exogenous peptide 25 enhanced T cell responses, indicating that adoptively-transferred P25TCRTh1 cells are capable of responding to antigen stimulation in the lungs, it was hypothesized that availability and/or presentation of antigen is a limiting factor in the activation of CD4⁺ effector T cells at the site of M. tuberculosis infection. To test this hypothesis, it was first investigated whether changes in the expression of the M. tuberculosis gene that encodes Ag85B influence the frequency of activation of P25TCRTh1 effector cells. It was found that the frequency of in vivo activation of P25TCRTh1 cells mimicked the temporal pattern of expression of fbpB (which encodes Ag85B) by M. tuberculosis in vivo (FIG. 3C). This suggests that reduced expression of Ag85B contributes to the low frequency of activation of Ag85B-specific CD4⁺ effector cells in the lungs, thus resembling previously-reported observations with Salmonella FliC expression and FliC-specific CD4⁺ T cell responses (Cummings et al., “FliC-Specific CD4+ T Cell Responses are Restricted by Bacterial Regulation of Antigen Expression,” J. Immunol. 174:7929-7938 (2005), which is hereby incorporated by reference in its entirety).

### Example 8

### Forced Expression of fbpB Induces Greater P25TCRTh1 Cell Activation

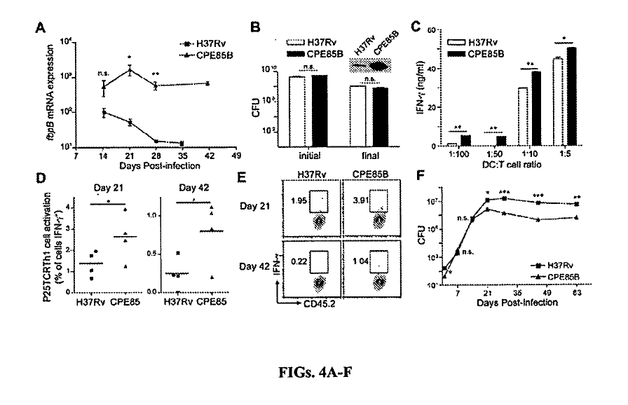

To test the hypothesis that fbpB down-regulation contributes to the submaximal frequency of CD4⁺ effector cell activation and the limited efficacy of the Th1 response in vivo, a recombinant strain of M. tuberculosis was constructed to express fbpB at high levels during chronic infection. Using the ΔAg85B strain as a background, a wild-type fbpB allele was introduced under control of the hspX/acr/Rv2031c promoter to the M. tuberculosis chromosome via the pMV306 integrating vector. hspX is expressed at high levels during chronic phase infection in an expression pattern inverse to fbpB (Banaiee et al., “Regulation of Mycobacterium tuberculosis whiB3 in the Mouse Lung and Macrophages,” Infect. Immun. 74:6449-6457 (2006); Shi et al., “Expression of Th1-Mediated Immunity in Mouse Lungs Induces a Mycobacterium tuberculosis Transcription Pattern Characteristic of Nonreplicating Persistence,” Proc. Natl. Acad. Sci. 100:241-246 (2003), which are hereby incorporated by reference in their entirety). This strain (hspXp:fbpB, termed “CPE85B” for chronic phase expressed Ag85B) exhibited higher fbpB expression compared to H37Rv in the lungs of mice after aerosol infection (FIG. 4A). The expression of fbpB measured by RTqPCR was approximately 10-fold higher (normalized for the abundance of 16S rRNA) at day 21 post-infection for CPE85B than for H37Rv. As the infection progressed to chronic phase (day 28-42 post-infection), fbpB expression from the native promoter declined by approximately 100-fold while fbpB expression driven by the hpsX promoter remained at nearly constant, higher levels (FIG. 4A). Increased fbpB gene expression in the CPE85B strain was accompanied by markedly enhanced expression and secretion of Ag85B protein when the hspX promoter was induced in stationary liquid culture (FIG. 4B).

To determine whether forced expression of fbpB in M. tuberculosis results in increased presentation of Ag85B peptide 25 to CD4⁺ T cells, bone marrow-derived dendritic cells (BMDC) were infected with either H37Rv or CPE85B and compared their ability to activate P25TCRTh1 cells in culture. At all APC:T cell ratios examined, DCs infected with CPE85B induced significantly greater amounts of IFN-γ secretion from P25TCRTh1 cells than did DCs infected with H37Rv (FIG. 4C). To determine whether forced expression of fbpB can increase the frequency of P25TCRTh1 stimulation during H37Rv infection in vivo, the frequency of P25TCRTh1 cell activation in the lungs of mice infected with either H37Rv or CPE85B were compared. Compared to cells from H37Rv-infected recipients, P25TCRTh1 cells from CPE85B-infected mice produced IFN-γ with a 2-fold (day 21) to 5-fold (day 42) higher frequency (FIGS. 4D and E). These findings indicate that forced expression of fbpB by M. tuberculosis increases the proportion of P25TCRTh1 cells that are activated to produce IFN-γ in the lungs. By suppressing fbpB expression after the initial stages of infection, wild-type M. tuberculosis can reduce the frequency of activation of Ag85B-specific effector T cells. Although expression of fbpB was maintained at high levels from day 14 to day 42 post-infection, P25TCRTh1 cell stimulation in CPE85B-infected mice was only two- to five-fold higher than in mice infected with H37Rv, and decreased as infection progressed to chronic stage, indicating that other mechanisms, such as inhibition of antigen presentation and/or induction of regulatory T cells, exist to limit the activation of CD4⁺ effector T cells in the lungs.

### Example 9

### Forced Expression of fbpB Impairs Bacterial Persistence During Chronic Infection

It was reasoned that, if diminishing fbpB expression during chronic infection limits effector T cell activation and thereby enables M. tuberculosis to evade adaptive immunity, then constitutive expression of fbpB throughout infection should improve immune control of infection. To test this hypothesis, mice were infected with either H37Rv or CPE85B and quantitated M. tuberculosis CFUs in the lungs throughout the course of infection. The rates of bacterial growth for the two strains were indistinguishable prior to day 14 post infection (FIG. 4F), indicating that expression of fbpB by the hspX promoter does not attenuate M. tuberculosis in vivo during the innate immune stage of infection, prior to recruitment of CD4⁺ effector T cells to the lungs. Indeed, the in vivo generation time of the CPE85B strain (23.0 h) was slightly shorter than that of H37Rv (26.4 h) during days 1-14 of infection (these are not significantly different by nonlinear curve fit and F test). However, at times corresponding to the adaptive immune phase of infection, the bacterial burden of the CPE85B strain in the lungs was approximately 10-fold lower than that of H37Rv (FIG. 4F). These results suggest that forced expression of fbpB partially overcomes the antigen deficit that limits the activation of CD4⁺ T cells in the lung during chronic infection and allows greater antimycobacterial efficacy of the adaptive immune response.

### Example 10

### Chronic Phase Attenuation of CPE85B is Dependent on CD4+ T Cells

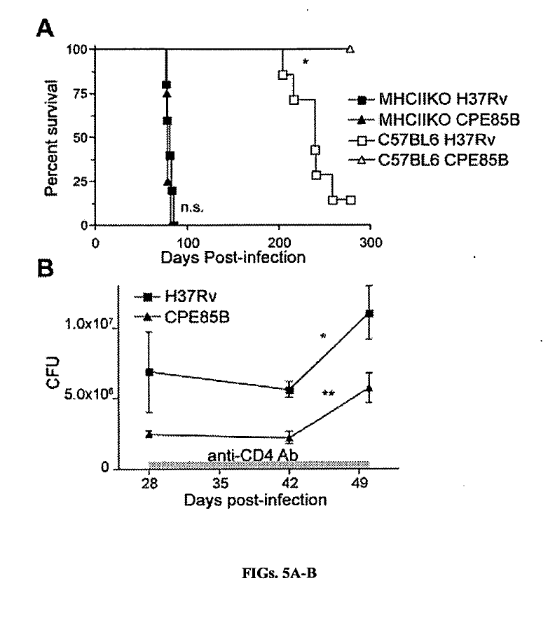

The observation that CPE85B demonstrates a growth pattern indistinguishable from H37Rv during the first two to three weeks of infection, prior to onset of adaptive immunity, suggested that CPE85B was not inherently attenuated for growth in vivo. However, the possibility that over-expression of fbpB could cause attenuation of M. tuberculosis as a result of gene dysregulation or toxicity of an overabundant Ag85B protein was considered. Notably, CPE85B demonstrated a similar growth pattern to H37Rv during in vitro shaking culture. Furthermore, under conditions of hypoxic stationary culture, when Ag85B protein is strongly expressed by CPE85B compared to H37Rv, the survival of the CPE85B strain is not impaired compared with that of wild-type bacteria (FIG. 4B). Taken together, these findings imply that impaired persistence of M. tuberculosis CPE85B in vivo is the consequence of increased antigen presentation and activation of CD4⁺ T cells, and not due to intrinsic attenuation of the CPE85B strain in vitro or in vivo. It was reasoned that if the decreased lung bacterial burden of CPE85B compared with that of H37Rv is attributable to increased antigen presentation and recognition by CD4⁺ T cells, then the attenuated phenotype of CPE85B should be abrogated in mice lacking CD4⁺ T cells. Indeed, whereas wild-type C57BL/6 mice infected with CPE85B survived significantly longer than those infected with H37Rv (median survival >300 and 239 days, respectively; p=0.0062), MHCIIKO mice, which lack CD4⁺ T cells, exhibited indistinguishable susceptibility to infection with the CPE85B and H37Rv strains (median survival 79 and 81 days, respectively; p=0.425), (FIG. 5A), clearly establishing that in vivo attenuation of the CPE85B strain depends on MHC II antigen presentation and CD4+ T cell responses. These results also indicate that increased antigen expression, accompanied by increased antigen-specific T cell activation, can enhance control of M. tuberculosis without detectable detrimental effects, since wild-type mice infected with the CPE85B strain survived longer than mice infected with H37Rv.

Since MHC II-deficient mice are highly susceptible to M. tuberculosis infection, this could potentially mask any hypothetical CD4⁺ T cell-independent mechanisms of attenuation of the CPE85B strain. It was reasoned that, if mechanisms other than increased CD4⁺ T cell recognition contribute to the lower burdens of CPE85B, then this strain would not recover and grow normally in the lungs when CD4⁺ T cells are depleted during the chronic phase of infection. Mice were infected with H37Rv or CPE85B. The infection was allowed to proceed for 28 days, when initial lung CFUs were measured for each group. As expected, bacterial CFUs for CPE85B were ˜3 fold lower than H37Rv at this time point (FIG. 5B). The remaining mice in each infection group were then treated with monoclonal antibody GK1.5 every 6 days until day 50 post-infection to deplete CD4⁺ T cells. After an initial lag, in which neither bacterial strain expanded, both CPE85B and H37Rv resumed growth in the lungs at indistinguishable rates (FIG. 5B). Taken together, these data provide strong evidence that improved control of the CPE85B strain is attributable to increased activation of Ag85B-specific CD4⁺ T cells, although the possibility that other factors contribute to the lower lung burdens of CPE85B that appear after the development of adaptive immunity cannot be excluded.

### Example 11

### Treatment with Intravenous Peptide 25 During Chronic Infection Reduces Lung Bacterial Burden

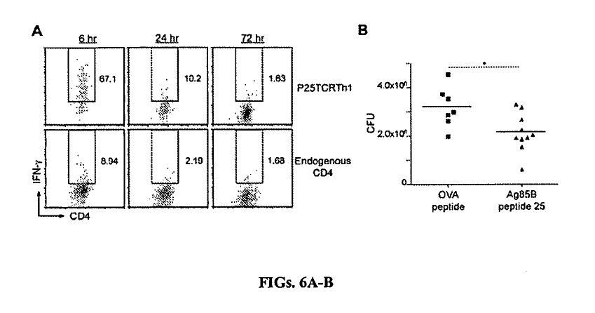

The observation that forced expression of fbpB increased the frequency of Ag85B peptide 25-specific CD4⁺ T cells and reduced the bacterial burden in the lungs (FIGS. 4D, 4E, and 4F), together with the observation that injection of peptide 25 also increased activation of CD4⁺ effector T cells at the site of infection (FIGS. 2B and 2C) suggested that providing antigen by injection of peptide 25 might also result in improved immune control of infection. The duration of increased IFN-γ production by adoptively transferred P25TCRTh1 cells or endogenous CD4⁺ T cells was first determined after peptide 25 injection. The frequency of IFN-γ cells was highest in both 6 hours after treatment, and decreased to approximately 20% of maximal levels by 24 hours after peptide injection for both endogenous CD4+ and P25TCRTh1 cells (FIG. 6A). By 72 hours post-treatment, the frequency of IFN-γ⁺ cells returned to levels observed in the absence of peptide 25 injection, indicating that the activating effect of peptide 25 treatment is remarkably transient, entirely dissipating within 3 days of the treatment. Despite the transient nature of this effect, it was found that treatment of M. tuberculosis H37Rv-infected mice with peptide 25 (in the absence of adoptively transferred P25TCRTh1 cells) every 2-3 days from day 28 to day 45 post-infection reduced lung bacterial burdens by 1.0560.406106 bacteria (p=0.018) compared with that in mice treated with OVA peptide, an unrelated MHC II epitope (FIG. 6B). Neither group of mice displayed any signs of toxicity, even after repeated peptide injections. These results indicate that during M. tuberculosis infection, CD4⁺ effector T cells are not stimulated at their maximum potential frequency at the site of infection in the lungs. Because effector T cell responses progressively decrease during chronic infection, and enhancing T cell responses with exogenous peptide antigen improves immune clearance of M. tuberculosis, it was concluded that failure to optimally activate effector T cells at the site of infection is an important determinant of the limited efficacy of adaptive immunity in tuberculosis.

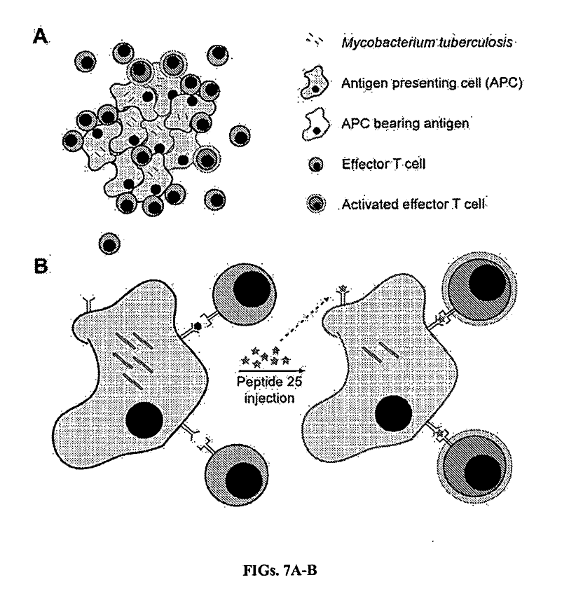

M. tuberculosis evades adaptive immunity to persist in the lungs, often for the lifetime of the host. Here, one mechanism by which this impressive feat of immune evasion is accomplished in vivo has been characterized. It was found that, of the large number of CD4⁺ effector T cells recruited to the lungs of infected mice, few are stimulated to produce IFN-γ (FIG. 7A). While there are few precedents available for comparison, the findings are in stark contrast to those found in C57BL/6 mice infected with the Armstrong strain of LCMV (Liu & Whitton, “Cutting Edge: Re-Evaluating the In Vivo Cytokine Responses of CD8+ T Cells During the Primary and Secondary Viral Infections,” J. Immunol. 174:5936-5940 (2005), which is hereby incorporated by reference in its entirety). In that context, which results in CD8⁺ T cell-dependent resolution of infection, >20% of virus specific CD8⁺ T cells are activated to produce IFN-γ during the acute stage of infection when viral burdens and antigen availability are highest, and the frequency of in vivo-activated virus-specific CD8⁺ T cells does not decrease until the viral burden is reduced. It was found that the initially low proportion of CD4⁺ T cells producing IFN-γ in the lungs of M. tuberculosis-infected mice diminishes further as infection progresses to chronic phase, even though the bacterial burden in the lungs remains high. The studies using adoptively transferred Ag85B-specific P25TCRTh1 cells revealed that the decreasing responses of CD4⁺ effector cells are caused in part by decreasing expression of fbpB by M. tuberculosis. By reducing fbpB expression during chronic infection, M. tuberculosis restricts the availability of Ag85B, an immunodominant antigen, and thereby prevents infected APCs from optimally activating CD4⁺ effector T cells. Consistent with this model, it was found that a recombinant strain of M. tuberculosis engineered to maintain the expression of fbpB at high levels during chronic infection (CPE85B) was attenuated during the chronic phase of infection in a strictly CD4⁺ T cell dependent manner, indicating that down-regulation of fbpB and limitation of antigen availability is important for evasion of adaptive immunity by M. tuberculosis. Treatment of infected mice with synthetic Ag85B peptide 25 also increased CD4⁺ effector T cell IFN-γ responses and significantly reduced the bacterial burden in the lungs. It was concluded that suboptimal effector T cell activation enables M. tuberculosis to evade elimination by adaptive immunity during the chronic stage of infection, and that some of this suboptimal effector T cell activation is attributable to restricted antigen expression by the bacteria. In addition, other mechanisms that limit effector T cell activation, such as interference with the MHC class II antigen processing and presentation pathway and/or the action of regulatory T cells, likely contribute to the remarkable survival of M. tuberculosis in vivo.

Infection with M. tuberculosis induces a robust T cell response involving CD4⁺ and CD8⁺ T cells and the effector cytokines IFN-γ and TNF (Cooper, “T Cells in Mycobacterial Infection and Disease,” Curr. Opin. Immunol. 21:378-384 (2009), which is hereby incorporated by reference in its entirety), which are all essential for control of infection (Flynn et al., “Tumor Necrosis Factor-Alpha is Required in the Protective Immune Response Against Mycobacterium tuberculosis in Mice,” Immunity 2:561-572 (1995); Mogues et al., “The Relative Importance of T Cell Subsets in Immunity and Immunopathology of Airborne Mycobacterium tuberculosis Infection in Mice,” J. Exp. Med. 193:271-280 (2001), which are hereby incorporated by reference in their entirety), yet adaptive immunity fails to eradicate M. tuberculosis. Mechanisms for the limited efficacy of the adaptive immune response in tuberculosis fall into two general (not mutually exclusive) categories: Either the effector functions that T cells perform (e.g. IFN-γ production) are not effective because of failed responses by the infected cells targeted by effector T cells; or the T cells recruited to the site of infection do not optimally perform the effector functions required for immune clearance. Regarding the former, the ability of M. tuberculosis to resist and inhibit the TNF and IFN-γ-induced microbiocidal responses of the phagocytic cells it infects is one documented component of its immune evasion strategy in vivo (Jung et al., “Virulent but not Avirulent Mycobacterium tuberculosis can Evade the Growth Inhibitory Action of a T Helper 1-Dependent, Nitric Oxide Synthase 2-Independent Defense in Mice,” J. Exp. Med. 196:991-998 (2002), which is hereby incorporated by reference in its entirety). However, the observation that only a small fraction of the CD4⁺ effector T cells in the lungs is activated to synthesize IFN-γ provides new support for the latter explanation. The potential causes of this mechanism include bacterial factors and host regulatory mechanisms that directly impair effector T cell function. As an example of a direct bacterial effect, mycobacterial cell wall glycolipids have been found to impair CD4⁺ T cell responses in vitro (Mahon et al., “Mycobacterium tuberculosis Cell Wall Glycolipids Directly Inhibit CD4+ T-Cell Activation by Interfering with Proximal T-Cell-Receptor Signaling,” Infect. Immun. 77:4574-4583 (2009), which is hereby incorporated by reference in its entirety). With regard to host regulatory mechanisms, during mouse infection, T regulatory cells limit the ability of adaptive immunity to restrict the bacterial population size in the lungs (Scott-Browne et al., “Expansion and Function of Foxp3-Expressing T Regulatory Cells During Tuberculosis,” J. Exp. Med. 204:2159-2169 (2007); Shafiani et al., “Pathogen-Specific Regulatory T Cells Delay the Arrival of Effector T Cells in the Lung During Early Tuberculosis,” J. Exp. Med. 207:1409-1420 (2010), which are hereby incorporated by reference in their entirety). Interleukin-10 (“IL-10”), whether expressed by myeloid cells or T cells, provides an additional host regulatory mechanism that inhibits T cell effector functions in tuberculosis, as transgenic over-expression of IL-10 in infected mice impaired T cell responses and caused an increase in bacterial CFUs (Turner et al., “In vivo IL-10 Production Reactivates Chronic Pulmonary Tuberculosis in C57BL/6 Mice,” J. Immunol. 169:6343-6351 (2002), which is hereby incorporated by reference in its entirety), while deletion of IL-10 causes enhanced control of infection (Redford et al., “Enhanced Protection to Mycobacterium tuberculosis Infection in IL-10-Deficient Mice is Accompanied by Early and Enhanced Th1 Responses in the Lung,” Eur. J. Immunol. 40:2200-2210 (2010), which is hereby incorporated by reference in its entirety), indicating that T cell-directed suppressive factors can limit the success of the adaptive immune response to M. tuberculosis. On the other hand, CD4⁺ effector T cells at the site of infection may not recognize or become activated optimally by APCs bearing M. tuberculosis-derived peptide:MHC II complexes, a process that is required for IFN-γ production in peripheral tissues (McLachlan et al., “Dendritic Cell Antigen Presentation Drives Simultaneous Cytokine Production by Effector and Regulatory T Cells in Inflamed Skin,” Immunity 30:277-288 (2009), which is hereby incorporated by reference in its entirety). Recent observations using live imaging revealed that a small fraction of Leishmania major-infected macrophages interact with Leishmania specific CD4⁺ T cells in vivo (Filipe-Santos et al., “A Dynamic Map of Antigen Recognition by CD4 T Cells at the Site of Leishmania Major Infection,” Cell Host Microbe 6:23-33 (2009), which is hereby incorporated by reference in its entirety) indicating that in certain infections, effector T cells may not recognize infected cells efficiently, and this may contribute to slow clearance or persistence of infection. Suboptimal stimulation of CD4⁺ T cells could occur via direct targeting and inhibition of MHC II antigen presentation pathways in infected APCs, or as a result of the limited availability of peptide T cell epitopes, a consequence of bacterial suppression of antigen encoding genes, or a combination of these mechanisms.

In this study, it was first determined that the frequency of endogenous polyclonal CD4⁺ T cells producing IFN-γ in the lungs was surprisingly low, and varied during the course of infection, with the highest responses during the acute stage and the lowest responses observed as infection reached the chronic stage. These reduced responses occur despite the presence of similar numbers of bacteria in the lungs during these stages of infection. To further understand the underlying mechanisms of the low frequency of effector T cell activation in the lungs, CD4⁺ effector T cell responses were quantitated to the peptide 25 epitope of M. tuberculosis Ag85B, a secreted protein targeted by a large number of M. tuberculosis-specific CD4⁺ T cells (Rogerson et al., “Expression Levels of Mycobacterium tuberculosis Antigen-Encoding Genes Versus Production Levels of Antigen-Specific T Cells During Stationary Level Lung Infection in Mice,” Immunology 118:195-201 (2006), which is hereby incorporated by reference in its entirety). Ag85B is targeted by 5 of the 9 novel tuberculosis vaccine candidates currently in clinical trials (Barker et al., “Tuberculosis Vaccine Research: The Impact of Immunology,” Curr. Opin. Immunol. 21:331-338 (2009), which is hereby incorporated by reference in its entirety). Thus, understanding its behavior and responses to it in vivo has considerable importance for TB vaccine development. The reduced expression of fbpB observed is consistent with regulation by the state of bacterial growth, though it may be indirectly triggered by the onset of Th1 immunity, since expression of fbpB is maintained in mice lacking IFN-γ (Shi et al., “Expression of Th1-Mediated Immunity in Mouse Lungs Induces a Mycobacterium tuberculosis Transcription Pattern Characteristic of Nonreplicating Persistence,” Proc. Natl. Acad. Sci. 100:241-246 (2003), which is hereby incorporated by reference in its entirety). Because Ag85B is a cell wall biosynthesis enzyme, down-regulation of fbpB has been interpreted as a consequence of transition by M. tuberculosis into a relatively stationary state. Alternatively, fbpB suppression during chronic infection may also be an evolved bacterial immune evasion mechanism that enables long-term persistence of M. tuberculosis by limiting T cell activation. In support of this, it was found that forced expression of fbpB by the CPE85B strain during chronic infection resulted in a higher proportion of P25TCRTh1 cells producing IFN-γ than in H37Rv-infected mice. Other studies have suggested but not directly examined the possibility that over expression of certain M. tuberculosis proteins (including Hsp70 and ESAT-6) may cause attenuation of bacterial persistence by increased immune recognition (Stewart et al., “Overexpression of Heat-Shock Proteins Reduces Survival of Mycobacterium tuberculosis in the Chronic Phase of Infection,” Nat. Med. 7:732-737 (2001); Ohol et al., “Mycobacterium tuberculosis MycP1 Protease Plays a Dual Role in Regulation of ESX-1 Secretion and Virulence,” Cell Host Microbe 7:210-220 (2010), which are hereby incorporated by reference in their entirety). The finding that polyclonal CD4⁺ effector T cell responses diminish in chronic infection suggests that this may be a general phenomenon in tuberculosis. Importantly though, the higher frequency of P25TCRTh1 cell activation observed in CPE85B-infected mice diminished at a later time point as it did in H37Rv infection, implying that other mechanisms, especially impairment of MHC II antigen presentation by M. tuberculosis, exist to limit effector T cell activation during chronic infection in vivo.

Several in vitro studies have found that M. tuberculosis subverts or impairs antigen presentation by the cells it infects, limiting the capability of infected APCs to activate antigen specific T cells (Baena & Porcelli, “Evasion and Subversion of Antigen Presentation by Mycobacterium tuberculosis,” Tissue Antigens 74:189-204 (2009); Harding & Boom, “Regulation of Antigen Presentation by Mycobacterium tuberculosis: A Role for Toll-like Receptors,” Nat. Rev. Microbiol. 8:296-307 (2010), which are hereby incorporated by reference in their entirety). Initial observations include the finding that M. bovis BCG survives in primary human macrophages that CD4⁺ T cells fail to recognize (Pancholi et al., “Sequestration from Immune CD4+ T Cells of Mycobacteria Growing in Human Macrophages,” Science 260:984-986 (1993), which is hereby incorporated by reference in its entirety) and that M. tuberculosis-infected THP-1 cells express low amounts of surface MHC II (Hmama et al., “Attenuation of HLA-DR Expression by Mononuclear Phagocytes Infected with Mycobacterium tuberculosis is Related to Intracellular Sequestration of Immature Class II Heterodimers,” J. Immunol. 161:4882-4893 (1998), which is hereby incorporated by reference in its entirety). Several mechanisms for inhibition of MHC II antigen presentation have been characterized using a spectrum of mycobacterial strains and cell components. Among these, impaired phagosome maturation, a well-characterized component of the ability of M. tuberculosis to survive in phagocytic cells (Clemens & Horwitz, “Characterization of the Mycobacterium tuberculosis Phagosome and Evidence that Phagosomal Maturation is Inhibited,” J. Exp. Med. 181:257-70 (1995), which is hereby incorporated by reference in its entirety), has been found to limit activation of cathepsin D for efficient processing of mycobacterial antigens (Singh et al., “Processing and Presentation of a Mycobacterial Antigen 85B Epitope by Murine Macrophages is Dependent on the Phagosomal Acquisition of Vacuolar Proton ATPase and in situ Activation of Cathepsin D,” J. Immunol. 177:3250-3259 (2006), which is hereby incorporated by reference in its entirety), while inducing autophagy with rapamycin was recently found to improve the efficacy of BCG and other live mycobacterial vaccines, by enhancing presentation of mycobacterial antigens (Jagannath et al., “Autophagy Enhances the Efficacy of BCG Vaccine by Increasing Peptide Presentation in Mouse Dendritic Cells,” Nat. Med. 15:267-276 (2009), which is hereby incorporated by reference in its entirety). Impaired expression of MHC II by macrophages after IFN-γ treatment was also observed after in vitro infection or treatment of macrophages with certain mycobacterial cell components (Noss et al., “Toll-Like Receptor 2-Dependent Inhibition of Macrophage Class II MHC Expression and Antigen Processing by 19-kDa Lipoprotein of Mycobacterium tuberculosis,” J. Immunol. 167:910-918 (2001); Gehring et al., “Mycobacterium tuberculosis LprG (Rv1411c): A Novel TLR-2 Ligand that Inhibits Human Macrophage Class II MHC Antigen Processing,” J. Immunol. 173:2660-2668 (2004); Pecora et al., “Mycobacterium tuberculosis LprA is a Lipoprotein Agonist of TLR2 that Regulates Innate Immunity and APC Function,” J. Immunol. 177:422-429 (2006); Kincaid et al., “Mycobacterium tuberculosis Exerts Gene-Selective Inhibition of Transcriptional Responses to IFN-Gamma without Inhibiting STAT1 Function,” J. Immunol. 171:2042-2049 (2003); Fortune et al., “Mycobacterium tuberculosis Inhibits Macrophage Responses to IFN-Gamma through Myeloid Differentiation Factor 88-Dependent and -Independent Mechanisms,” J. Immunol. 172:6272-6280 (2004), which are hereby incorporated by reference in their entirety). This effect may involve prolonged signals received through bacterial pattern recognition receptors (PRRs) including TLR2, although a TLR2 independent mechanism for impaired MHC II expression in response to IFN-γ was recently reported (Fortune et al., “Mycobacterium tuberculosis Inhibits Macrophage Responses to IFN-Gamma through Myeloid Differentiation Factor 88-Dependent and -Independent Mechanisms,” J. Immunol. 172:6272-6280 (2004); Kincaid et al., “Codominance of TLR2-Dependent and TLR2-Independent Modulation of MHC Class II in Mycobacterium tuberculosis Infection In vivo,” J. Immunol. 179:3187-3195 (2007), which are hereby incorporated by reference in their entirety).

These and other in vitro studies are consistent with the present results and lend support for the hypothesis that APCs do not efficiently stimulate CD4⁺ effector T cells in the lungs during M. tuberculosis infection in vivo. Attempts to verify and explore the significance of these in vitro findings with in vivo infection models have been limited thus far, until the present study. One study of mouse infection with GFP-expressing M. bovis BCG found a modest decrease in surface expression of MHC II on some populations of lung APC that harbored intracellular bacteria when compared to those that did not contain bacteria (Pecora et al., “Mycobacterium bovis BCG Decreases MHC-II Expression In vivo on Murine Lung Macrophages and Dendritic Cells During Aerosol Infection,” Cell Immunol. 254:94-104 (2009), which is hereby incorporated by reference in its entirety). In contrast, in a low dose aerosol infection of mice with GFP-expressing H37Rv, a difference in surface MHC II expression was not detected between infected and non-infected APCs at various time points post-infection. It was also found that M. tuberculosis infected APCs isolated from the lungs expressed high levels of the costimulatory molecules CD80 and CD86 (Kincaid et al., “Codominance of TLR2-Dependent and TLR2-Independent Modulation of MHC Class II in Mycobacterium tuberculosis Infection In vivo,” J. Immunol. 179:3187-3195 (2007), which is hereby incorporated by reference in its entirety). Nonetheless, there is evidence that the activation of M. tuberculosis-specific T cell responses is impaired during in vivo infection, indicating that M. tuberculosis may specifically impair presentation of its antigens without decreasing overall surface expression of MHC II. One recent study found that mice provided with CD4⁺ TCR-transgenic effector T cells specific for the M. tuberculosis antigen ESAT-6 prior to infection can restrict bacterial population size to a lower level but cannot prevent establishment of infection (Gallegos et al., “Delayed Protection by ESAT-6-Specific Effector CD4+ T Cells After Airborne M. tuberculosis Infection,” J. Esp. Med. 205:2359-2368 (2008), which is hereby incorporated by reference in its entirety). Despite the presence of this effector T cell population in the lungs from the onset of infection, control of bacterial growth was delayed until 7 days post-infection. Likewise, despite mounting apparently normal anti-M. tuberculosis CD4⁺ T cell responses, infected mice and humans treated with anti-mycobacterial drugs to eliminate primary infection remain susceptible to reinfection (Jung et al., “Properties and Protective Value of the Secondary Versus Primary T Helper Type I Response to Airborne Mycobacterium tuberculosis Infection in Mice,” J. Exp. Med. 201:1915-1924 (2005); Chiang et al., “Exogenous Reinfection in Tuberculosis,” Lancet Infect. Dis. 5:629-636 (2005), which are hereby incorporated by reference in their entirety). These studies indicate that susceptibility to persistent tuberculosis is more likely due to failure to activate antigen-specific effector T cells, rather than to insufficient development of antigen specific T cells in response to infection.

An increased survival of wild-type, but not CD4⁺ T cell-deficient mice infected with the CPE85B strain was observed when compared to those infected with H37Rv, highlighting the importance of enhanced T cell stimulation to the long-term outcome of infection, and indicating that enhanced effector T cell activation, through increased antigen availability, can be accomplished without detrimental effects. Moreover, the finding that sustained expression of Ag85B during the adaptive immune phase of infection was associated with a 2- to 5-fold increase in antigen specific CD4⁺ T cell activation, yet reduced the bacterial burdens approximately 10-fold implies that a massive increase in effector T cell activation is not necessary to significantly improve immune control of tuberculosis. Future efforts to develop tuberculosis therapies should therefore aim to bypass or overcome factors that limit effector T cell activation including direct T cell suppression, impaired antigen presentation, and bacterial gene regulatory mechanisms. For example, it was found that the chronic phase antigen deficit resulting from bacterial suppression of fbpB could be overcome by systemic treatment of infected mice with synthetic peptide 25, which strongly but transiently enhanced CD4⁺ T cell responses specific for this epitope and reduced the bacterial burden. This result implies that the endogenous CD4⁺ T cells generated in response to infection with M. tuberculosis and recruited to the infected lungs can be stimulated to perform their effector functions if they are provided antigen, resulting in improved bacterial clearance (FIG. 7B). The potential for anti-tuberculosis therapies that aim to enhance existing T effector cell responses in infected individuals with synthetically produced peptides encoding known T cell epitopes remains unexplored. However, given the steadily increasing prevalence of drug resistant M. tuberculosis, such immunotherapeutic approaches to tuberculosis are an attractive option. Although the consequences of increasing the activation of existing T cell responses have not been widely tested, in the context of certain highly monoclonal T cell responses, administration of epitope peptides has caused rapid mortality of infected or previously immunized mice (Kitamura et al., “Long Peptide Vaccination Can Lead to Lethality through CD4+ T Cell-Mediated Cytokine Storm,” J. Immunol. 185:892-901 (2010); Liu et al., “Peptide Vaccination of Mice Immune to LCMV or Vaccinia Virus Causes Serious CD8 T Cell-Mediated, TNF-Dependent Immunopathology,” J. Clin. Invest. 116:465-475 (2006), which are hereby incorporated by reference in their entirety). However, despite these findings and concerns about possible immunopathology induced by hyperactivation of effector T cells in tuberculosis (Barber et al., “CD4 T Cells Promote Rather than Control Tuberculosis in the Absence of PD-1-Mediated Inhibition,” J. Immunol. 186:1598-1607 (2011), which is hereby incorporated by reference in its entirety), no morbidity or mortality was observed in infected mice repeatedly treated with peptide 25, a result that encourages the continued exploration of this therapeutic strategy. Future studies should also aim to determine the host and bacterial regulatory mechanisms that account for chronic phase suppression of fbpB and whether genes encoding other immunodominant M. tuberculosis antigens behave similarly. Identification of the elements of this host/pathogen interaction may lead to the development of therapies that target antigen gene suppression and inhibition of antigen presentation and provide a novel strategy for overcoming bacterial persistence in vivo, leading to better outcomes in M. tuberculosis infected individuals.

Although the invention has been described in detail for the purposes of illustration, it is understood that such detail is solely for that purpose, and variations can be made therein by those skilled in the art without departing from the spirit and scope of the invention which is defined by the following claims.

What is claimed:

1. A method of treating a subject for an infectious disease, said method comprising:
selecting a subject infected with an infectious disease-causing agent, wherein the infectious disease-causing agent elicits, in the selected subject, an immune response that is insufficient to cure the selected subject of the infectious disease and
administering to the selected subject a synthetic peptide representing an antigen epitope associated with an immune response to the infectious agent, wherein the immune response to the synthetic peptide is sufficient to treat the selected subject for the infectious disease.

2. The method according to claim 1, wherein the infectious disease is a chronic infectious disease.

3. The method according to claim 1, wherein the infectious disease is tuberculosis.

4. The method according to claim 3, wherein the antigen epitope is bound to an MHC protein on a surface of an antigen-presenting cell.

5. The method according to claim 3, wherein in response to said administering, cells in the selected subject's lungs are activated.

6. The method according to claim 1, wherein in response to said administering, CD4⁺ and CD8⁺ T cells are activated.

7. The method according to claim 1, wherein the antigen epitope is an epitope of an immunodominant antigen.

8. The method according to claim 1, wherein the antigen epitope is an epitope of a subdominant antigen.

9. The method according to claim 1, wherein the subject is a human subject.

10. The method according to claim 1, wherein the subject has a normal immune system.

11. The method according to claim 1, wherein said administering is carried out systemically.

12. The method according to claim 1, wherein the immune response from said administering kills the infectious disease-causing agent.

13. The method according to claim 12, wherein the infectious disease-causing agent is a bacterium.

14. The method according to claim 1, wherein the subject is infected with a multidrug-resistant and/or extensively drug-resistant strain of the infectious disease-causing agent.

15. The method according to claim 1, wherein the synthetic peptide is peptide 25 of SEQ ID NO:10.

16. A peptide comprising an antigen epitope associated with an immune response to Mycobacterium tuberculosis, wherein the peptide elicits an immune response in a subject that reduces bacterial burden in the subject beyond what a normal immune response in the subject can reduce.
